(19) United States
(12) Patent Application Publication
(10) Pub. No.: US 2011/0190793 A1
(43) Pub. Date: Aug. 4, 2011

(54) METHODS AND APPARATUSES FOR SUTURING OF CARDIAC OPENINGS

(75) Inventors: Anthony A. Nobles, Fountain Valley, CA (US); Benjamin G. Brosch, Mission Viejo, CA (US)

(73) Assignee: MED-VENTURE INVESTMENTS, LLC, Fountain Valley, CA (US)

(21) Appl. No.: 13/016,897

(22) Filed: Jan. 28, 2011

Related U.S. Application Data
(60) Provisional application No. 61/299,910, filed on Jan. 29, 2010.
(60) Provisional application No. 61/304,381, filed on Feb. 12, 2010.

(51) Int. Cl. A61B 17/04

(52) U.S. Cl. 606/144

(57) ABSTRACT

A suturing apparatus configured to suture an opening extending through an outer wall of the heart, such as a transapical opening, can comprise an elongate body having a proximal end, a distal end, a tapered or rounded tip, one or more arms, and one or more needles. A handle at the proximal end of the elongate body can be configured to be manipulated from outside of the heart. The elongate body can have a substantially constant outer diameter between the handle and the distal end. The arms can hold portions of suture a distance away from the outer diameter of the elongate body. The arms can be extendable from said body from a retracted position to an extended position. The arms in the extended position can point distally and form an acute angle with a longitudinal axis of the elongate body. The plurality of needles can be movable to pass through heart tissue into engagement with the suture portions held by the arms and back through the heart tissue to draw the suture portions through the heart tissue.

### CROSS-REFERENCE TO RELATED APPLICATIONS

This application claims the benefit of U.S. Provisional Application No. 61/299,910, which was filed on Jan. 29, 2010, and U.S. Provisional Application No. 61/304,381, which was filed on Feb. 12, 2010. The entirety of each of the priority applications is hereby incorporated herein by reference.

### BACKGROUND FIELD OF DISCLOSURE

The present disclosure relates in general to suturing devices and methods, and in certain embodiments to closure of openings in the outer wall of the heart, and, in some embodiments, more specifically to closure of transapical openings.

### BACKGROUND OF THE DISCLOSURE

Various medical procedures can be performed on the heart. These include treatments of atrial openings, septal defects, valves and other parts of the heart. Such procedures can be preformed percutaneously or laparascopically among other methods, to gain access to the heart. In some instances, the interior of the heart is accessed through a puncture or other passageway made through the heart wall tissue. FIG. 1 illustrates access to the heart through the apex 7 of the heart. For example, direct access may be gained to the left ventricle 6 through the apex 7.

During transapical delivery, the patient may receive a small puncture in the chest cavity where the operator can access the apex of the heart. In some instances, transapical access may be used to perform a procedure on a beating heart.

### SUMMARY

After a procedure on the heart through the heart wall is completed, the access hole in the heart, e.g., the apex, will desirably be closed using embodiments described further below. This closure can be particularly challenging in a beating heart procedure. The disclosed methods preferably effectively and efficiently close a transapical opening. Although embodiments are described herein in the context of transapical closure, some embodiments of the invention can be utilized for closing any suitable opening in tissue, and can have particular utility in closing openings used to provide access to the heart.

Accordingly, there is a need for an improved method to close a transapical and other openings during medical procedures performed on the heart. Methods and devices are described herein for closing punctures, incisions, and other openings within biological tissue.

In one embodiment, a device and method are provided for suturing biological tissue, such as, for example, the external heart wall. The device is particularly well suited to the anatomy of the heart for suturing an incision made in the apex of the heart, following a heart procedure. In some embodiments, the device can eliminate the need to apply pressure to a patient's heart for an extended period of time, and eliminates or reduces many complications and costs associated with such a procedure. In some embodiments, the device can comprise an improved handle portion that facilitates suture placement in a quick and efficient manner. Some embodiments of the handle portion are very simple to operate, thereby reducing or eliminating the possibility of human error during use. In some embodiments, actuation mechanisms of the handle portion facilitate maintenance of the device in a steady position while applying suture, particularly during a beating heart procedure.

In some embodiments, a suturing apparatus configured to suture an opening made through an outer wall of the heart is provided, comprising an elongate body having a proximal end, a distal end, a tapered or rounded tip positioned distally of the distal end of the elongate body and configured to be delivered through the opening and into an interior of the heart, and a handle at the proximal end of the elongate body configured to be manipulated from outside of the heart. The elongate body can have a substantially constant outer diameter between the handle and the distal end. The suturing apparatus can include a plurality of arms proximal to the tip arranged symmetrically about the outer diameter of the elongate body. The plurality of arms can be configured to hold portions of suture a distance away from the outer diameter of the elongate body. The plurality of arms can be extendable from said body from a retracted position to an extended position. The plurality of arms in the extended position can point distally and can form an acute angle with a longitudinal axis of the elongate body. The suturing apparatus can include a plurality of needles slidably housed in said elongate body. The plurality of needles can be movable along the longitudinal axis of the elongate body and outwardly from the body to pass through heart tissue into engagement with the suture portions held by the plurality of arms. The plurality of needles can be retractable away from the plurality of arms back through the heart tissue to draw the suture portions through the heart tissue.

In some embodiments, methods of closing a transapical opening in a wall of the heart are provided. In some embodiments, the disclosed methods can comprise delivering a suturing device through the transapical opening, where the suturing device comprises an elongate body having a proximal end and a distal end. At least one arm can be extended from the suturing device from a retracted position to an extended position. The at least one arm can hold a portion of suture. At least one needle can be advanced through heart tissue adjacent the transapical opening into engagement with the suture portion held by the arm. The at least one needle can be retracted through the heart tissue adjacent the transapical opening to draw the suture portion through the heart tissue. The transapical opening can be closed with the suture portion.

### BRIEF DESCRIPTION OF THE DRAWINGS

FIG. 1 is a schematic partial cross-sectional view of an exemplifying treatment area, comprising a human heart.

FIG. 2A is a perspective view of a portion of a suturing device in accordance with an embodiment.

FIG. 2B is a partial cross-sectional view of a handle of the suturing device of FIG. 2A.

FIG. 2C is a perspective view of a portion of the handle of the suturing device of FIGS. 2A and 2B.

FIG. 2D is a perspective partial cross-sectional view of the handle of FIGS. 2A-C.

FIGS. 3A-3C are perspective views of a distal end of the suturing device of FIG. 2A.

FIG. 3D is a perspective view of a distal end of a suturing device according to an embodiment.

FIG. 3E is a partial cross-sectional view of a portion of the distal end of a suturing device according to an embodiment.

FIG. 4A is a plan view of an embodiment of a suturing device.

FIG. 4B is a perspective view of the distal end of the suturing device of FIG. 4A.

FIGS. 4C is a partial cross-sectional view of the suturing device of FIGS. 4A and 4B.

FIG. 5 is a perspective view of a distal end of an embodiment of a suturing device.

FIGS. 6A-6C are partial cross-sectional views illustrating a method of placing suture through a heart wall according to an embodiment using the suturing device of FIGS. 6A-6C.

FIG. 7 is a cross-sectional view of a suture extending through tissue adjacent a transapical opening.

FIGS. 8-10 are cross-sectional views illustrating a method of placing suture through a heart wall according to an embodiment using the suturing device of FIGS. 4A-4C.

FIG. 11 is a cross-sectional view of sutures placed adjacent a transapical opening by the suturing devices of FIGS. 6A-6C and FIGS. 4A-4C.

FIG. 12 is a perspective view of an embodiment of a suturing device.

FIG. 13 is a perspective view of a distal end of the suturing device of FIG. 12.

FIG. 14 is a partial cross-sectional view of the suturing device of FIGS. 12 and 13 placing a suture adjacent a transapical opening.

FIG. 15 is a side perspective view of an embodiment of a suturing device.

FIGS. 16A-16B are partial cross-sectional views of the suturing device of FIG. 15.

FIG. 17 is a partial cross-sectional view of the suturing device of FIGS. 15 and 16 placing suture adjacent a transapical opening.

FIG. 18 is a partial cross-sectional view of pledget that has been placed over a suture closing a transapical opening.

FIG. 19 is a perspective view of a suture arm of an embodiment of a suturing device.

FIG. 20 is a side perspective view of an embodiment of a suturing device.

FIGS. 21A-21B are partial cross-sectional views illustrating a method of placing suturing adjacent a transapical opening using the suturing device of FIG. 20.

FIG. 22 is a partial cross-sectional view of an embodiment of a balloon used in connection with an embodiment of a suturing device.

FIG. 23 is a partial cross-section view of an embodiment of a balloon used in connection with an embodiment of a suturing device.

FIG. 24 is a side view of a knot placement device.

FIG. 25 is a cross-sectional view of a knot and knot placement device.

FIG. 26 is a cross-sectional view of an embodiment of a knot and knot placement device.

### DETAILED DESCRIPTION OF THE EXEMPLIFYING EMBODIMENTS

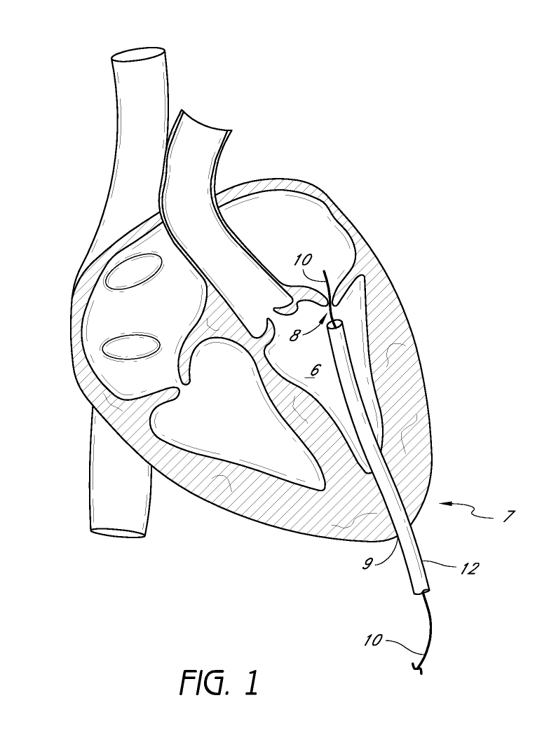

FIG. 1 illustrates access through the apex 7 of the heart. As depicted in FIG. 1, a guidewire 10 is advanced into the left ventricle 6 of the heart through a puncture or opening 9 near an apex 7. The heart may be accessed through a limited thoracotomy, small trocar puncture, or small catheter puncture. With the guidewire 10 in place, the physician can insert a sheath 12 to the left ventricle 6. Treatment devices can be advanced through the lumen of the sheath 12. For example, treatment devices may be delivered to mitral valve 8 to repair or replace the valve. In an alternative embodiment, devices can be advanced over the guidewire 10 and positioned at or near a desired location without the need to insert an introducer sheath 12.

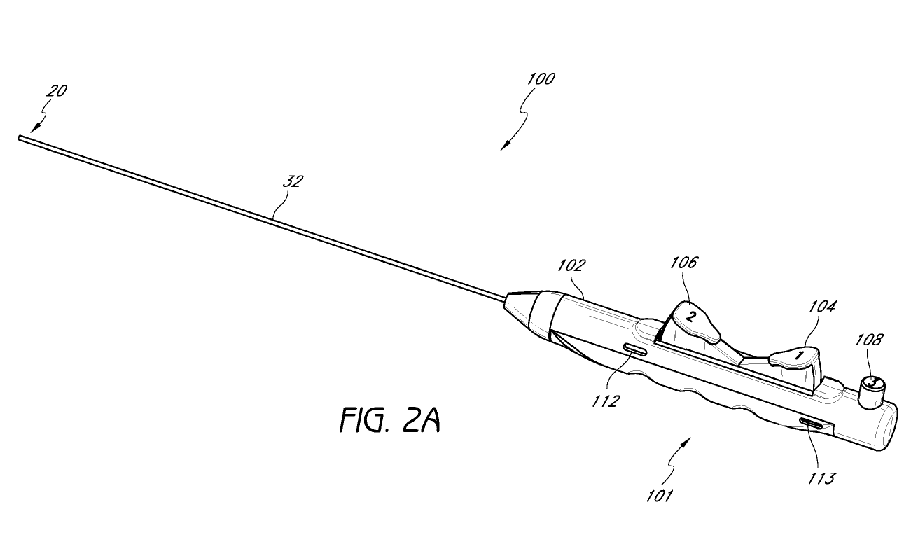

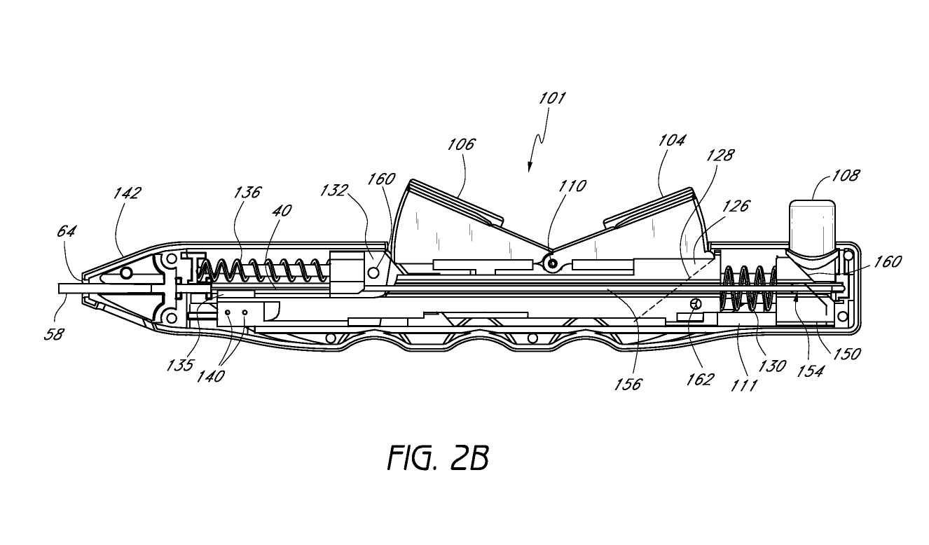

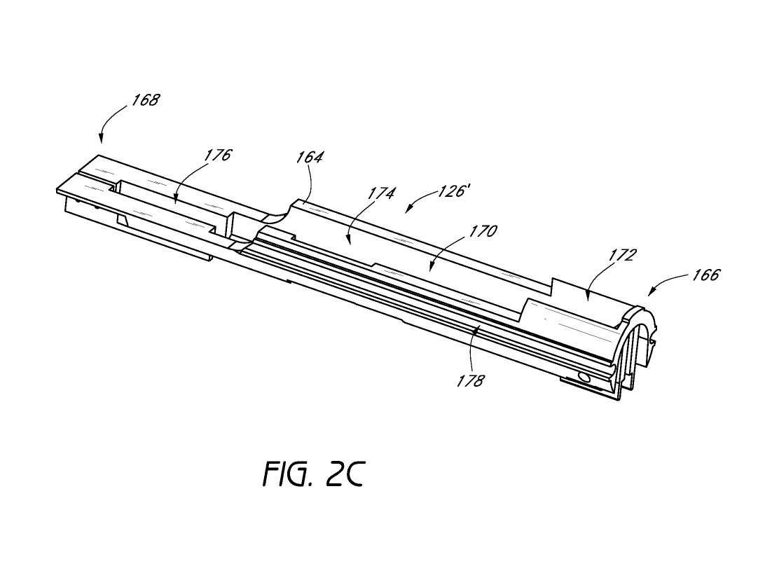

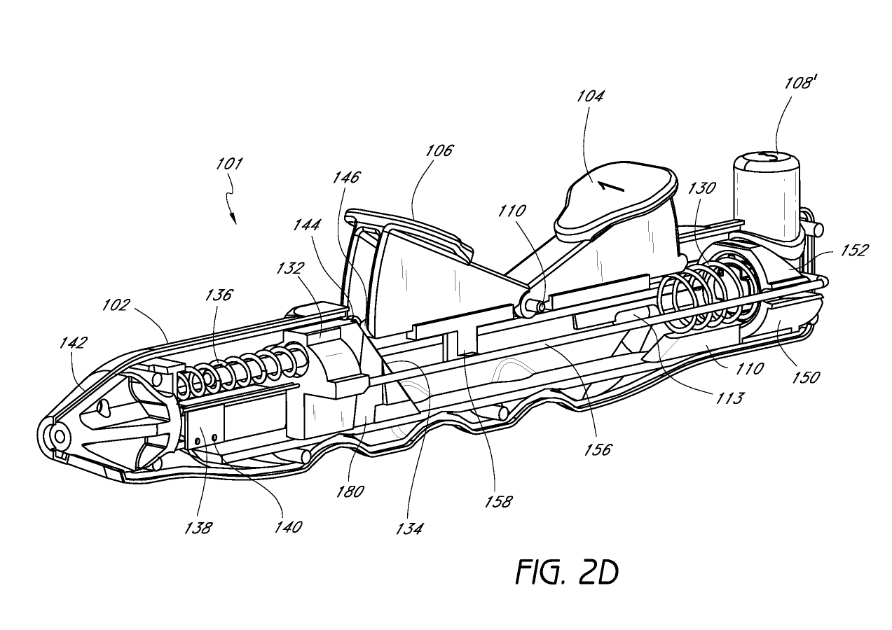

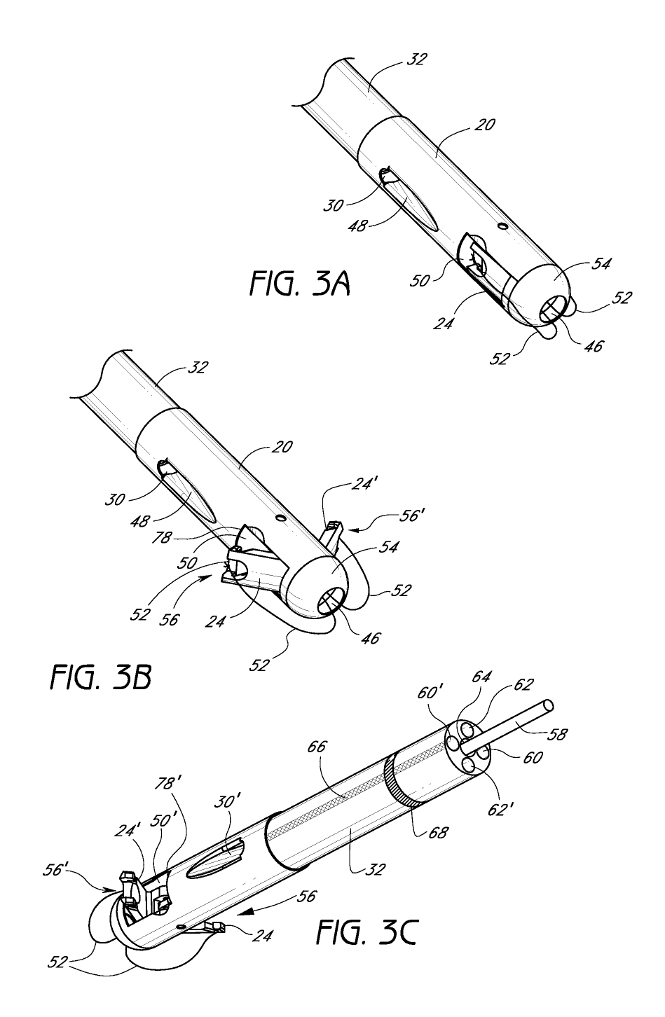

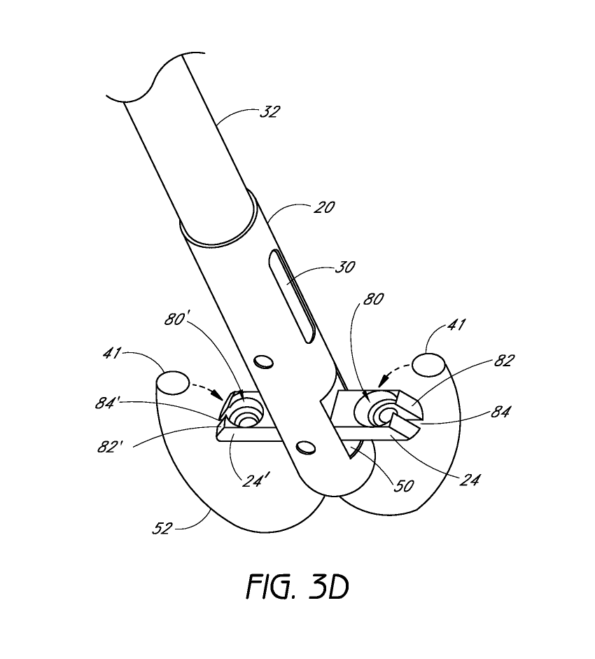

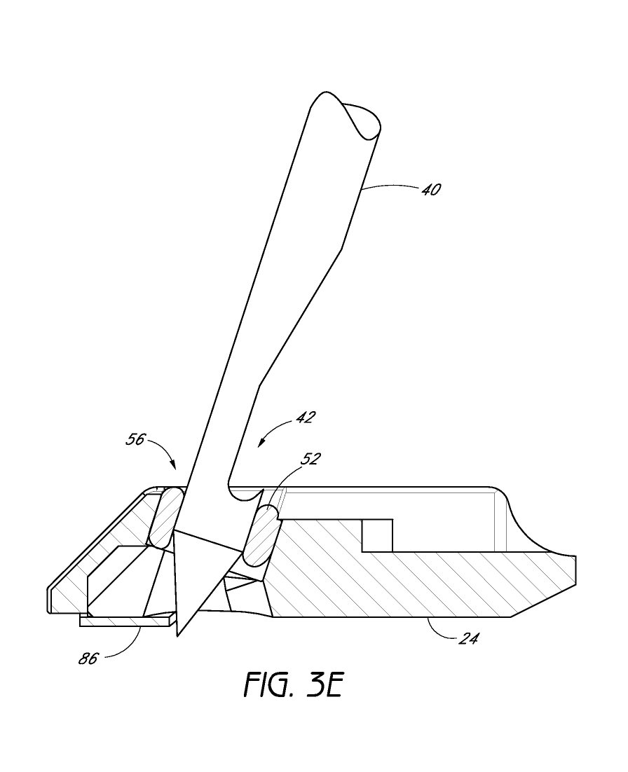

After the treatment of the heart is complete, a suitable suturing device, such as those disclosed herein, can be delivered through the apex to close the opening in the apex. In some embodiments, the introducer sheath 12 and/or guidewire 10 may be used to position the device within the opening 9. Alternatively, the suturing device may be directly inserted into the opening 9. FIGS. 2-3 illustrate one embodiment of a suturing device 100 that can be used to place suture through heart tissue to close the opening in the apex.

In the embodiment illustrated in FIG. 2A, the suturing device 100 generally comprises an elongate body 32, a distal end 20, and a handle portion 101. The handle 101 comprises actuators 104, 106, 108. The handle portion 101 advantageously allows the physician to operate the suturing apparatus such that suture may be applied to an opening in a very quick and easy manner. The handle portion advantageously requires little manipulation during use. In some embodiments, the handle can be operated with a single hand. The suturing apparatus can be used to close an opening located deep within the patient's tissue (e.g., the heart) without requiring the application of pressure over an extended period of time. As a result, the suturing apparatus can substantially reduce the recovery period following a medical procedure, thereby allowing the patient to return home more quickly and substantially reducing costs. The dimensions of the suturing device 100 can vary according to the approach to the transapical site and the particular medical procedure performed. In one configuration, the suture distal end 20 and the hollow elongate body 32 have constant diameters of about 6-16 Fr and a length of about 15 to 80 cm, more preferably less than about 80 cm, 70 cm, 60 cm, 50 cm, 40 cm or 30 cm. In some embodiments, the elongate body 32 has a constant diameter and the distal end 20 can taper toward the distal end to a smaller diameter. In some embodiments, both the elongate body 32 and the distal end 20 can include selectively tapering portions along the longitudinal direction.

As shown in FIG. 2A, the handle portion 101 comprises a main housing 102, an arm trigger 104, a needle trigger 106, and an arm release button 108. The arm and needle triggers can provide actuators for producing movement of internal components within the main housing, which in turn move at least one arm 24 (FIG. 3A-C) and needle 40 (FIG. 3E) for applying suture to a treatment site. As will be described in more detail below, the handle portion can be constructed such that the arm trigger 104, needle trigger 106 and arm release button 108 can be depressed by the physician in a particular order to extend and retract cooperating suture arms and needles along the distal end 20 for applying suture to a transapical opening. The arm trigger 104, needle trigger 106 and arm release button 108 can include markings to indicate the order in which the triggers are actuated, e.g., the arm trigger 104 can be labeled “1,” the needle trigger 106 can be labeled “2,” and the arm release button 108 can be labeled “3.”

As illustrated in FIG. 2B, the arm and needle triggers can be pivotally coupled to the main housing 102 about pin 110 such that the triggers rotate as they are depressed by the physician. As will be described in more detail below, the pivotal rotation can facilitate the cam-like interaction of the triggers with the internal components of the main housing. When the arm trigger 104 is depressed, it pivots about a distal end, and engages an inclined surface 128 of a first follower member 126. The first follower member 126 can be slidably received in the main housing 102 and can include an elongate body 164 (FIG. 2C) having a proximal end 166 and a distal end 168, with a slot 170 extending longitudinally through the elongate body 164 along a top side thereof. At the proximal end 166, the elongate body 164 can have a partially circular cross-section, with a proximal portion 172 of the slot receiving the arm trigger 104 (FIGS. 2A-2B) when depressed. The inclined surface 128 (FIG. 2B) can be provided within the proximal portion 172 of the slot 170 to engage the depressed arm trigger 104. An intermediate portion 174 of the elongate body 164 slot 170 can partially receive the needle trigger 106 when depressed. A distal portion 176 of the elongate body 164 slot 170 can partially receive the needle trigger 106 (FIG. 2B) when depressed, and also can receive a second follower member 132, as described further below. Along both sides of the outer surface of the elongate body 164 adjacent the slot 170 portions, longitudinal grooves 178 can be provided to receive an arm lockout wire 156, described further below.

A drive wire tab 138 can be secured to the distal end 168 of the first follower member 126, such as by pins through holes 140. The tab 138 can be secured to an actuating rod 58, which extends through a central lumen 64 of an extrusion clamp 142.

FIG. 2D illustrates the handle 101 with the first follower member 126 removed. A downwardly extending leg 158 can extend from a lower surface of the needle trigger 106. When the first follower member 126 is in its initial configuration, a ledge (not shown) on the first follower member can be positioned below the leg 158, preventing the needle trigger 106 from being depressed. When the first follower member 126 is moved proximally, the ledge can move proximally to allow downward movement of the needle trigger 106. This prevents the needle trigger 106 from being actuated until after the arms are deployed by depressing arm trigger 104.

The needle trigger 106, when depressed, engages an inclined surface 134 of the second follower member 132, causing the second follower member 132 to compress a needle biasing spring 136. The second follower member 132 can be provided in the distal portion 176 (FIG. 2C) of the slot 170 and can be capable of sliding relative to the first follower member 126. Proximal of the first follower member 126 can be an arm spring 130, and proximal of arm spring 130 can be third follower member 150, which has an inclined surface 152 (FIG. 2D) which can engage arm release button 108. Third follower member 150 can have longitudinal grooves 154 on both sides thereof to receive the arm lockout wire 156 described below. Elongate member 111 (FIG. 2B) can extend distally from the third follower member 150 underneath the arm spring 130.

As shown in FIG. 2B, the arm lockout wire 156 can extend proximally from one side of the second follower member 132, through the longitudinal groove 154 on one side of the elongate body 164 (FIG. 2C) of the first follower member 126, through the longitudinal groove 154 on one side of the third follower member 150, around the proximal end of the third follower member, and back through the grooves 154 and 178 on the opposite side of the housing and connecting with the second follower member 132. When second follower member 132 moves distally, arm lockout wire 156 can move distally, and become positioned underneath the arm release button 108. This can prevent the arm release button 108 from being depressed while the needles 40 are being actuated, until the second follower member 132 returns to its initial position.

The needle biasing spring 136 can engage the second follower member 132 for maintaining the second follower member 132 in the proximal position in the absence of any external input. Although one particular embodiment of a needle biasing spring 136 is shown for purposes of illustration, a wide variety of different biasing mechanisms can be used for biasing the second follower member 132 into the proximal position.

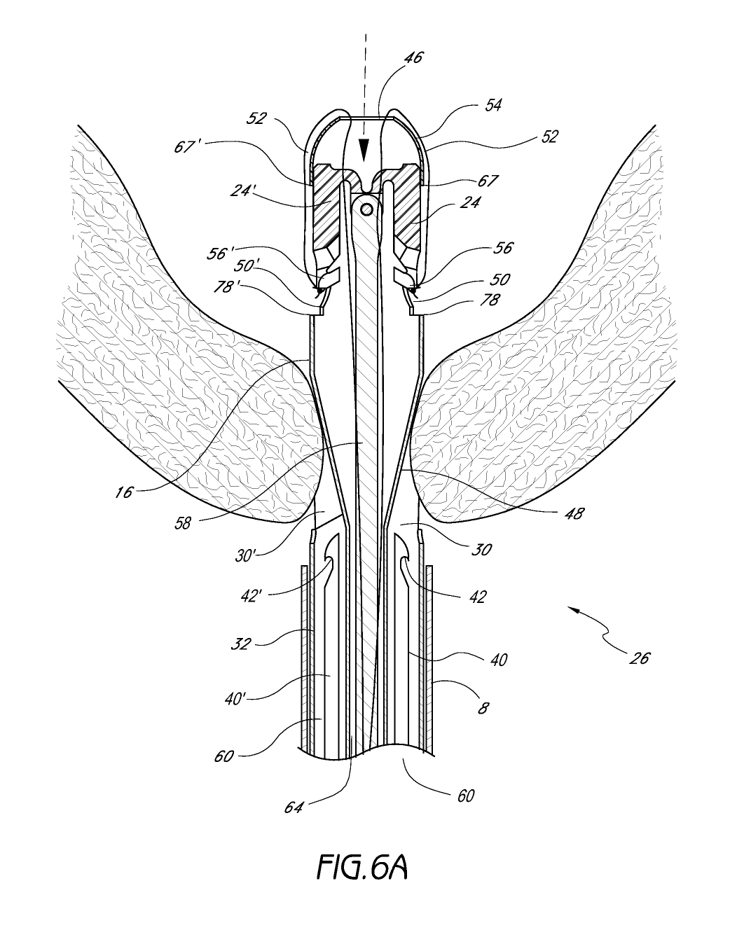

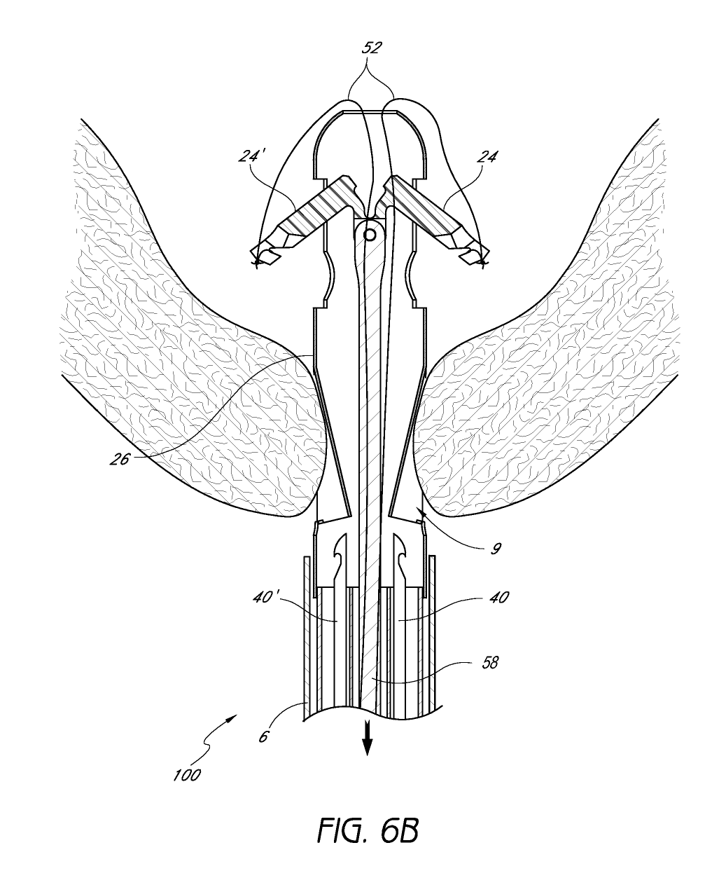

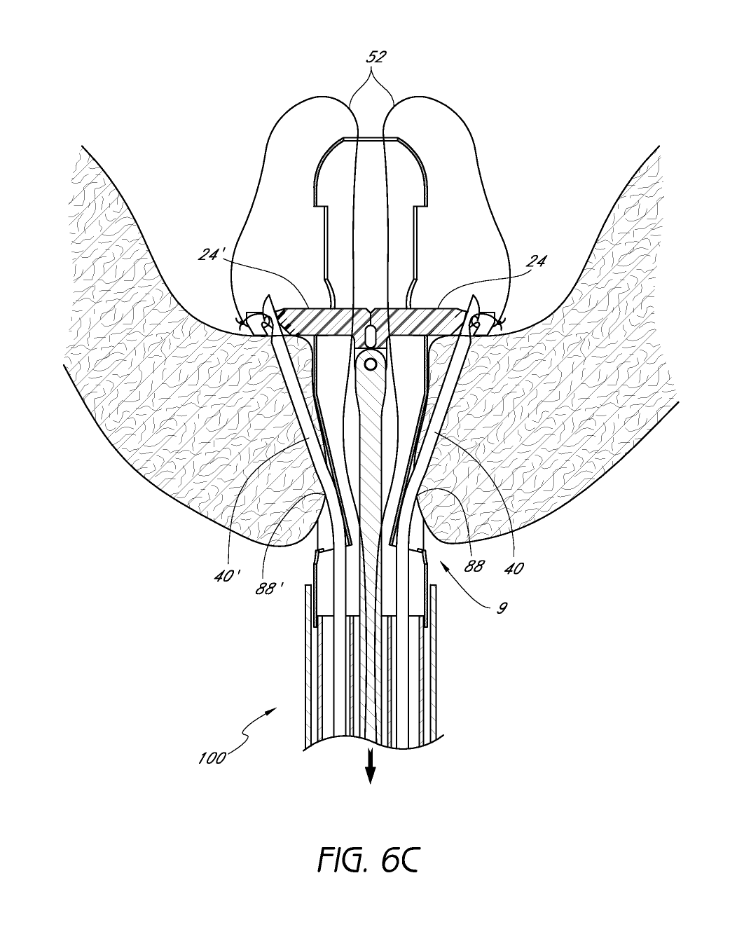

As the needle trigger 106 is depressed, the camming surface of the needle trigger 106 can push against the inclined surface 134 of the second follower member 132. The force from the needle trigger can impose a longitudinal force on the second follower member that causes the second follower member to slide distally relative to the main housing. As the second follower member translates distally within the housing, the needles 40 (FIG. 6C) can be pushed in a distal direction, thereby causing the distal end portions of the needles 40 to extend outward from the distal end 20 for engagement with the suture arms 24. The extension of the needles 40 from the distal end 20 will be described in more detail below. In some embodiments, the second follower member 132 can be contained within a body portion that is integral with the first follower member 126. The body portion provides a slotted track such that the second follower member 132 can be guided proximally and distally during use. Thus, the first and second follower members 126, 132 can be slidably coupled to each other.

It should also be noted that the second follower member 132 can be formed with a longitudinal lumen for slidably receiving the actuating rod 58. Accordingly, the actuating rod 58 can be slid longitudinally by movement of the first follower member 126 without interfering with the second follower member 132. The second follower member 132 can include an inclined surface 152. It can be seen that the lower portion of the second follower member 132 can be thinner in construction. The thinner section can be configured to fit within a groove in the body portion for guiding the movement of the second follower member 132, as described above.

As shown in FIG. 2A, the main housing 102 of the handle portion 101 can include a safety opening or window 112 to provide for manually retracting the needles in the event the needles become stuck in the tissue during retraction. This provides a safety mechanism to ensure that the needles of the suturing apparatus cannot become stuck in the extended position. The main housing 102 also can include a safety opening or window 113 to provide for manually retracting the arms. The opening 112 cooperates with an opening 160 in the second follower member 132, allowing for a pin, or tool, (not shown) to be inserted into the openings to apply force and manually bring the second follower member 132 and the needles back to its initial configuration. The opening 113 cooperates with the opening 162 in the first follower member 126 for the same purpose, to retract the arms to their initial configuration.

The inclined surfaces 128 (FIG. 2B), 134 (FIG. 2D) of the first and second follower members 126, 132 are shaped to produce a desired motion in response to actuation of the arm and needle triggers 104, 106, respectively. In one embodiment, at least a portion the inclined surface 134 of the second follower member 132 can be inclined at about 35° or more relative to the longitudinal axis. In another embodiment, at least a portion of the inclined surface 134 can be inclined at about 40° or more relative to the axis. In another variation, at least a portion of the inclined surface 134 can be inclined at about 41° relative to the axis. In another variation, at least a portion of the inclined surface 134 can be inclined at between about 35-45° relative to the axis. In another variation, at least a portion of the inclined surface 134 can be inclined at between about 39-43° relative to the axis. In another variation, at least a portion of the inclined surface 134 can be inclined at between about 40-42° relative to the axis. In still another variation, the inclined surface 134 can be curved. The same preferred ranges also apply to the inclined surface 128 of the first follower member 126. It will be appreciated that the ratio of trigger movement to needle movement can be proportional to the angle of the inclined surfaces 128, 134.

It has been found that the above angles provide excellent performance while minimizing the diameter of the handle portion. For example, a lower angle would make the follower members 128, 134 more difficult to move due to frictional forces. On the other hand, a higher angle would necessitate a larger follower member in order to produce the same amount of longitudinal translation, thereby necessitating a larger (e.g., larger diameter) handle portion. Furthermore, it has been found that an inclined surface 128, 134 formed with a substantially constant angle can provide a substantially directly proportional relationship between trigger movement and needle 40 movement. As a result, the physician can be able to advance and retract the needles 40 with great precision and predictability by controlling the movement of the needle trigger 106.

With reference again to FIGS. 2A-2D, the main housing 102 can be constructed of a translucent or transparent material, such as plastic, such that the movement of the components within the main housing can be visible to the physician. The transparency advantageously provides visual feedback to the physician regarding operation of the suturing apparatus. If desired, markings or other indicia can be provided such that the position of the needles 40 can be easily perceived during use. Alternatively, a window can be provided for observing the movement of the internal components or a portion of one or more internal components can extend through the main housing 102 to an exterior surface for purposes of visibility.

Operation of the suturing assembly 100 handle portion 101, as illustrated in FIGS. 2A-2D, can first begin, after appropriate placement of the assembly, by depressing arm trigger 104 labeled “1”. Depressing arm trigger 104 causes the first follower member 126 to move proximally within the housing 102, compressing arm spring 130 and moving the actuating rod 58 to deploy the arms 24. When the first follower member 126 can be in the distal position, the arms are fully contained within the distal end. However, when the first follower member 126 can be moved proximally by the arm trigger 104, each of the arms deploys outward through apertures on the sides of the distal end, as described in detail below. Accordingly, longitudinal movement of the first follower member 126 relative to the main housing controls the position of the arms. An arm spring 130 can provide a biasing force to maintain the first follower member 126 in the distal position in the absence of any external input. Although one type of arm spring 130 can be shown for purposes of illustration, any known biasing mechanisms can be used for maintaining the first follower member 126 into the distal position.

Arm trigger 104 can be depressed until it can be secured or locked in a down position, such that a shaped protrusion providing the camming surface can allow the arm trigger to be held in the deployed position. As a result, it is not necessary for the physician to apply a constant a force on the arm trigger 104 to maintain the suture arms in the deployed condition. The protrusion can be shaped to be captured and held beneath the first follower member 126 when the arm trigger 104 can be fully depressed. Accordingly, the cooperation of the protrusion and the first follower member 126 can create a detent mechanism such that the arm trigger 104 can be selectively maintained in the depressed position.

Next, depressing needle trigger 106 labeled “2” causes the second follower member 132 to slide distally within the slot of the first follower member 126, compressing the needle biasing spring 136 and causing needles 40 to splay outward from the elongate body 32 (FIG. 6C). More particularly, the needle trigger 106 can include features, e.g. pins, or the like, that ride initially along an inclined surface 134 of the second follower member 132, thereby causing the follower member to move in a distal direction for extending the needles. With respect to FIG. 2D, as the needle trigger 106 reaches a finish (i.e., fully depressed) position, the features, e.g. pins, extend beyond the bottom edge of the inclined surface 134, thereby relieving the force on the follower member and allowing the follower member to snap back in a proximal direction. This occurs while maintaining the needle trigger in the fully depressed condition. Accordingly, the needles are first fully extended and then automatically snap back when the needle trigger reaches a first finished position (i.e., can be fully depressed). When the needle trigger is released, the pins ride back up via slots 180 (FIG. 2D).

In the embodiment of FIGS. 2A-2D, the needle trigger can remain in its depressed configuration after the second follower member 132 snaps back to its original configuration, or the needle trigger can automatically return to its initial configuration. If the needle trigger 106 does not automatically return to its initial configuration, the operator can simply pull the needle trigger upward along the body of the second follower member until the riding features of the needle trigger 106 are once again above the inclined surface 134.

With respect to FIG. 2B, to retract the arms 24, the operator can press down on the arm release button 108, labeled “3”. This causes the third follower member 150 to move distally, and causes the elongate member 111 to contact a corner portion of the arm trigger 104 and urge the arm trigger distally so that it can be released from the first follower member 126.

With reference to FIGS. 3A-C, the device 100 can include one or more arms 24 at a distal end 20 of the constant diameter elongate body. In some embodiments, the device can include two, four, six, eight arms or more, or alternatively, an odd number of arms. The arms 24 can be moved between a retracted position, shown in FIG. 3A, and a deployed position. In some embodiments, the positions of the arms 24 in FIGS. 3B-3C can be a partially-deployed position. The arms 24 are configured to hold an end portion of a suture 52.

The device 100 can include one or more needles 40 (see FIG. 6C) that are movable between a retracted position, as in FIGS. 3A-C, and an advanced position, shown in FIG. 6C. In the advanced position, the needles 40 move outwardly away from the elongate body and intersect the deployed arms 24 to snatch the suture 52.

With reference to FIGS. 3A-3C, the distal end portion of the suturing apparatus will now be described in more detail. The illustrated distal end portion provides one embodiment that can be operated using the improved handle portion described above. As shown, the distal end portion can include the suture distal end 20, a pair of suture arms 24, 24′, a pair of suture clasps 56, 56′, a pair of suture arm apertures 50, 50′, a pair of curved or slanted needle guides 48, 48′, a pair of needle apertures 30, 30′, a distal end 54, a hole 46, a suture 52 and an actuating rod 58. The distal end portion can further include a pair of needles 40, 40′ (see FIGS. 6A through 6C). When the suture arms 24, 24′ are retracted into the suture arm apertures 50, 50′ and the needles 40, 40′ are retracted into the needle apertures 30, 30′, the arms 24, 24′ and the needles 40, 40′ are recessed within the distal end 20, as shown in FIG. 3A. This prevents the arms 24, 24′ and the needles 40, 40′ from causing tissue damage while the distal end portion passes through a biological structure.

FIGS. 3B and 3C illustrate the distal end portion of the suturing device 100 (FIG. 2A) with the suture arms 24, 24′ deployed outwardly from their recessed positions. Such deployment can be achieved by depressing the arm trigger 104, as described above with reference to FIGS. 2A through 2D. Depressing the arm trigger 104 translates the first follower member 126 and actuating rod 58 proximally, which brings the suture arms 24, 24′ into contact with a pair of proximal inside edges 78, 78′ of the suture arm apertures 50, 50′. As the arm trigger is depressed further, the proximal inside edges 78, 78′ force the suture arms 24, 24′ into a deployed state. The suture arms 24, 24′ extend a distance of about 1 mm to 15 mm away from the outer diameter of the elongate body 32. In one embodiment, the suture arms 24, 24′ continue to deploy radially until the arms 24, 24′ are approximately or substantially at 45° to the longitudinal axis of the suturing device 100, as shown in FIGS. 3B and 3C. In other embodiments, the suture arms 24, 24′ can be “fully” deployed when they reach an acute angle relative to the longitudinal axis of the distal end 20 or an obtuse angle relative to each other, for example about 20 to 70 degrees, more preferably about 30 to 60 degrees, even more preferably 40 to 50 degrees.

As illustrated in FIG. 3B, each of the suture arms 24, 24′ can include a suture clasp 56, 56′ which hold an end of the suture 52. Each of the suture arms 24, 24′ can be pre-loaded with the ends of the suture 52 before operation. The ends of the suture 52 can then pass from the suture clasps 56, 56′ to the distal hole 46 whereby the ends of the suture 52 enter the distal end 20 and can be passed proximally through the hollow elongate body 32. In the embodiment illustrated in FIG. 3B, each end of the suture 52 has a capture portion comprising a loop which can be tied onto the ends of the suture clasps 56, 56′. It is contemplated, however, that the capture portions are not restricted solely to tied loops, rather other types of capture portions can be utilized such as, by way of example, spheres or ferrules.

FIG. 3C illustrates one preferred configuration of the constant diameter elongate body 32 which can include five lumens. Two of the lumens 60, 60′ can be used to house the needles 40, 40′. Once the suture arms 24, 24′ have been deployed by depressing the arm trigger 104, as discussed with reference to FIGS. 3B-3C and in greater detail below, the needle trigger 106 can be depressed to advance the needles 40, 40′ (see FIGS. 6A-6C) from a recessed position within the distal end 20 to a distally extended position (see FIG. 6C). In one embodiment, the needles 40, 40′ move distally at substantially the same time. In other embodiments, the needles 40, 40′ can be actuated separately such that one of the needles 40, 40′ advances before the other.

When the two needles 40, 40′ move distally, the needle guides 48, 48′ direct the needles 40, 40′ out of the needle apertures 30, 30′ at an angle relative to the longitudinal axis of the distal end 20, as illustrated in FIG. 3C. The needles 40, 40′ can be flexible and made of a material with shape memory, such as SUPERFLEX NITINOL™. Alternatively, the needles 40, 40′ can be comprised of spring steel, surgical stainless steel or any variation thereof. Each of the needles 40, 40′ has a diameter of about 0.019 inches, but needles with other diameters can be used in accordance with the particular medical procedure contemplated.

When the needles 40, 40′ advance distally, as discussed above, the needle guides 48, 48′ cause the needles 40, 40′ to bend radially outward. As shown most clearly in FIG. 6C, a further outward, radial bend can be imparted to the needles 40, 40′ when they come into contact with a pair of annular recess 80, 80′ of the suture arms 24, 24′. When the needles 40, 40′ are retracted into the needle lumens 60, 60′, the needles 40, 40′ return to a straight configuration as a result of their resiliency. Although the embodiment of FIGS. 3A through 3C can include flexible needles 40, 40′, which bend during deployment, it is contemplated that other embodiments can advantageously comprise rigid needles which can be permanently straight or curved.

Referring again to FIG. 3C, the elongate body 32 contains a central lumen 64 which can be used to house the actuating rod 58. Another lumen 62 can be used to house the length of the suture 52 to prevent the suture 52 from becoming tangled. Alternatively, the suture 52 can be passed through the central lumen 64 along with the actuating rod 58.

In one embodiment, two thin stripes 66 (only one shown in FIG. 3C) marked on the exterior of the elongate body 32 extend along the entire length of the elongate body 32. The stripes 66 provide a visual indication of the circumferential location of the needles 40, 40′ relative to the elongate body 32. The stripes 66 facilitate aligning the needles 40, 40′ with the apex of the heart external wall surface, so that needle incisions 88, 88′ (FIG. 6C) formed in the heart wall tissue by the needles 40, 40′ will be aligned with the heart wall tissue. This enables the physician to place the suture 52 within the heart wall tissue such that the suture 52 closes the opening 9 transversely to heart internal wall cavity. This can be an efficient direction to close the opening 9. Proper insertion of the needles 40, 40′ reduces the risk of damage to the heart wall tissue. Alternatively, the elongate body 32 can have only one stripe 66 which denotes the circumferential location of one of the two needles 40, 40′. Because the needles 40, 40′ deploy from opposite sides of the distal end 20, knowledge of the location of one needle provides the physician with knowledge of the location of the other needle.

As illustrated in FIG. 3C, the exterior surface of the elongate body 32 can include a marker 68 which denotes a proximal position to which a sheath 12 should be partially withdrawn after the distal end 20 of the suturing device 100 has been inserted into the heart to expose the needle apertures 30, 30′. The partial withdrawal of the sheath 12 is discussed in detail in U.S. Pat. No. 6,562,052, entitled SUTURING DEVICE AND METHOD, the entirety of which is hereby incorporated by reference. The marker 68 can be shown as a visual marker, but can additionally or alternatively be in the form of a ridge, groove, or other physical structure which interacts with a corresponding structure of the sheath 12 to allow the physician to position the sheath 12 using a sense of feel. For example, the sheath 12 and the elongate body 32 could be configured to releasably engage or interlock with one another when the sheath 12 reaches a predetermined position along the elongate body 32. It is contemplated that a specially formed sheath 12 can include such an interlocking structure, and can be included within the scope of the invention. It is further contemplated that one or more additional markers (not shown) can advantageously be provided along the length of the elongate body 32, distal to the marker 68, to indicate other positions of the sheath 12 relative to the elongate body 32, such as the position at which the suture arms 24, 24′ can be exposed outside the sheath 12.

FIG. 3D illustrates a perspective view of the distal end 20 and the hollow elongate body 32 of an embodiment of the suture clasp arms 24, 24′. In this embodiment, the ends of the suture can be provided with special loops 41 that can be configured to engage with the needles, as described below.

As shown in FIG. 3D, arms 24 can include a hinge portion 636 and an annular recess 632 for holding a suture end loop 41 and for receiving the distal portion of a needle 40. The arms 24 can include a slit 640 for the length of the suture 52.

In one embodiment, the end loop 41 can include an eyelet that can be formed as a unitary, integral part of the suture 52. The suture eyelet can include a flat, thin portion of suture material having a central opening that can be slightly smaller than the base of the needle 40 tip. The periphery of the loop 41 can be contoured to match that of the recess 80 of the clasp arms 24. The loop 41 can be sized to fit within the recess 80 and to be retained therein by interference fit. The end loop 41 of the suture 52 can be formed by heating one end of a length of suture 52 such as by a stream of hot gas until the end becomes a ball-shape and pliable. The ball-shaped end can be then deformed by compressing it into a disc shape while the suture material 52 can be still pliable. A sharpened hypotube can then be used to punch out the hole near the center of the disc-shaped end such that the disc-shaped end forms the loop 41. If desired, the loop 41 can be bent relative to the strand while the material is pliable to put a permanent set in the bent suture. In some embodiments, the suture can include a monofilament or plastic suture material, such as prolene or declene. In one method of forming the end loop 41, instead of heating the end of a suture length, the suture end can be simply compressed and a hole can be formed thereafter. The end loop 41 can be cut or stamped into a circle shape.

In another embodiment, instead of pre-forming the hole in the suture end, the actuation of the needles 40 can be used to form the hole and fasten the ends of the suture 52 to the needles 40. In another embodiment, a separately-formed loop 41 can be insert-molded, glued, crimped or otherwise attached to the end of a length of suture 52. The loop 41 can be in the shape of a circle, oval, triangle, rectangle, hexagon, octagon, or the like.

The general use and operation of the suture clasp arms 24 in FIG. 3D can include placing the looped ends 41 of the suture 52 within the annular recess 80 of the suture clasp arms 24. The distal end 20 can be inserted into biological tissue and the suture clasp arms 24 can be deployed radially outward (FIGS. 6A-6C). The penetrating flexible needles 40 pass through the biological tissue to be sutured and engage the suture clasp arms 56.

When the needles 40 pass through the end loops 41 of the suture 52, the end loops 41 elastically stretch slightly, so as to circumferentially flex momentarily. As the needles 40 continue to advance distally, the end loops 41 relax, fall into the hook 42, and fasten around the needle grooves 42, such that pulling the needles 40 proximally causes the suture end loop 41 to follow the proximal movement of the needles 40. In an alternative embodiment, the needles can be formed without a groove or shoulder, and the shaft of the needle 40 can be sized relative to the opening in the eyelet to provide an interference fit therebetween.

The distal end of the angled needle guide 48 (FIG. 3A-3C) are preferably aligned with the suture arms 24, such that when the distal tip of the needle 40 is extended toward the arm 24, the needle 40 will be deflected toward the suture loops 41. Before insertion, the needle 40, has been advanced through the lumen 60 in the constant diameter elongate body 32 and positioned proximal to the needle aperture 30.

In use, when the distal end of the needle 40 is pushed through the needle guide 48 of the elongate body 32, the needle 40 will be advanced along the groove of the needle guide 48 and deflected outward along the angle path of the groove to penetrate the loop 41 on the arm 24 and engage suture portion 52 held therein. Once the needle 40 has engaged the suture portion 52, the distal end of the needle 40 can then be pulled proximally through the elongate body 32 which will cause the bent portion of the needle to be retracted along needle guide 48 into central lumen 60 along with the suture portion 52 held on the needle 40.

Under some circumstances, a hook 42, as illustrated on the needle 40 in FIG. 3E, when advanced can be oriented in a direction toward a location slightly closer to or slightly farther from the longitudinal axis of the elongate body 32 than the center of the suture clasp 56. A hook 42 can be oriented in a direction that can be toward a location slightly closer to or slightly farther from the longitudinal axis of the suturing device 100 than the center of the suture clasp 56 can, in some instances, not properly engage the suture. As a result, the suture catch mechanism, or the hook 42, may not successfully retract the suture end portion. The hook 42 can be oriented in any sufficient direction, e.g. radially outward from the elongate body 32 longitudinal axis, or the like. Additionally, the geometry of the hook 42 feature can be any geometry sufficient to snatch the suture loop 41, e.g. curved backward opposite the extension direction, sharp, blunt, extending around the full, or less than full, circumference of the needle 40, or the like.

In some embodiments, one or more of the suture arms 24 can have a deflector 86 (FIG. 3E). The deflecting plates 86 are shown partially obscuring an opening on a back side of the suture clasp 56 relative to the direction from which the needle hook 42 can penetrate the suture clasp 56. As the needle 40 passes through the suture clasp 56, the needle 40 can engage the deflector plate 86 and can be diverted from its previous course toward a longitudinal axis of the suturing device 100. In an alternative embodiment, the suture catch mechanism can be diverted away from a longitudinal axis of the device. As the needle 40 is diverted toward the longitudinal axis of the elongate body 32, the suture engaging portion, such as the hook 42, a recess, or a groove, can move toward a portion of a suture, shown schematically in FIG. 3E.

The dimensions of the needle 40 including the size and location of the suture engaging portion and the size and shape the needle 40 tip, the size of the suture clasp 56, and the distance by which the deflector plates obscure the back opening of the suture clasp 56 can be relatively proportioned such that the deflector 86 can urge the suture engaging portion of the needle 40, or the hook 42, to engage the portion of the suture end 52 as the needle 40 returns to its previous orientation. Thus, in the embodiment illustrated in FIG. 3E, the hook 42 can engage the suture portion 52 as the hook 42 is retracted from the suture clasp 56.

The deflector plates 86 can be generally rectangular, or can have other configurations, e.g. H-shaped, or the like. In some embodiments, the deflector plates 86 can be made from metal, while in other embodiments the deflector plates 86 can be made of plastics or other materials of sufficient rigidity or resiliency to deflect a suture catch mechanism. The deflector plates 86 can be joined to the suture clasp arms by welding, epoxy, adhesives or by other methods. In some embodiments, the deflectors 86 can be integrally formed with the suture clasp arm 24.

In some embodiments, the deflectors 86 can compensate for misalignment of a hook 42 with the center of a suture clasp 56 relative to a longitudinal axis of the suturing device 100 to provide consistent capture of a suture 52 end portion by the suture hook 42.

In some embodiments, bending of the constant diameter elongate body 32 can affect the relative position of the ends of the hook 42 and the distal end 20. For example, if the elongate body 32 is bent, then the distal end of the hook 42 extending along the inside of the bend in the elongate body 32 relative to the central axis of the elongate tubular member would be advanced relative to the spreader assembly. In such a circumstance, the hook 42 may be advanced through a suture clasp 56 farther than is desired, which may result in enlargement of a loop 41 at an end of a suture that in turn inhibits the ability of the hook 42 to retract the end of the suture 52.

On the other hand, if the elongate body 32 is bent, then the distal end of the hook 42 extending along the outside of the bend in the elongate body 32 relative to the central axis of the elongate body 32 would be retracted relative to the distal end 20. In such a circumstance, the hook 42 may not be advanced through the suture clasp 56 far enough to engage the suture end portion 52 held by the suture clasp 56.

In some embodiments, the effects of bending of the elongate tubular member can be reduced or eliminated by using a hook 42 that can be sufficiently long to engage the corresponding suture clasp 56 even if the hook 42 extends along the outside of a bend in the elongate body 32 relative to the central axis of the elongate body 32. Advancement of the suture catch mechanism can be limited by providing a stop mechanism in proximity to the distal end 20 where the effects of bending can be small or absent.

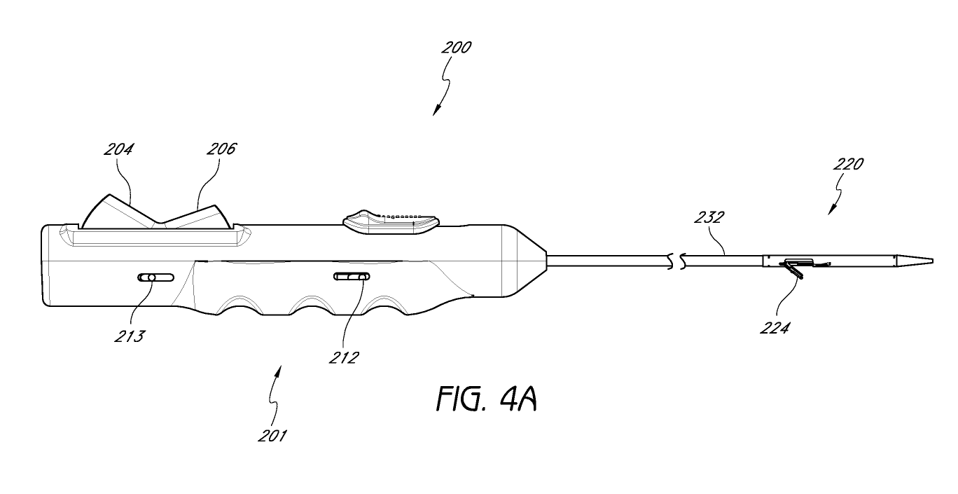

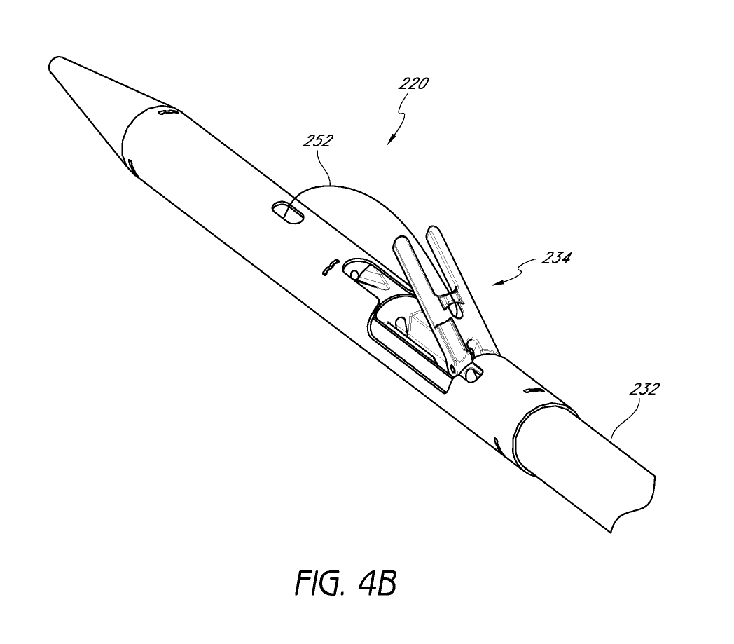

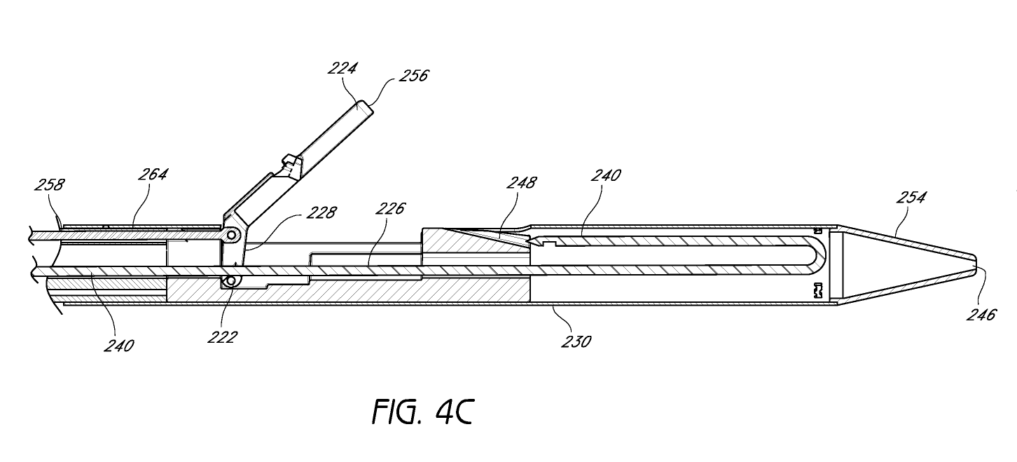

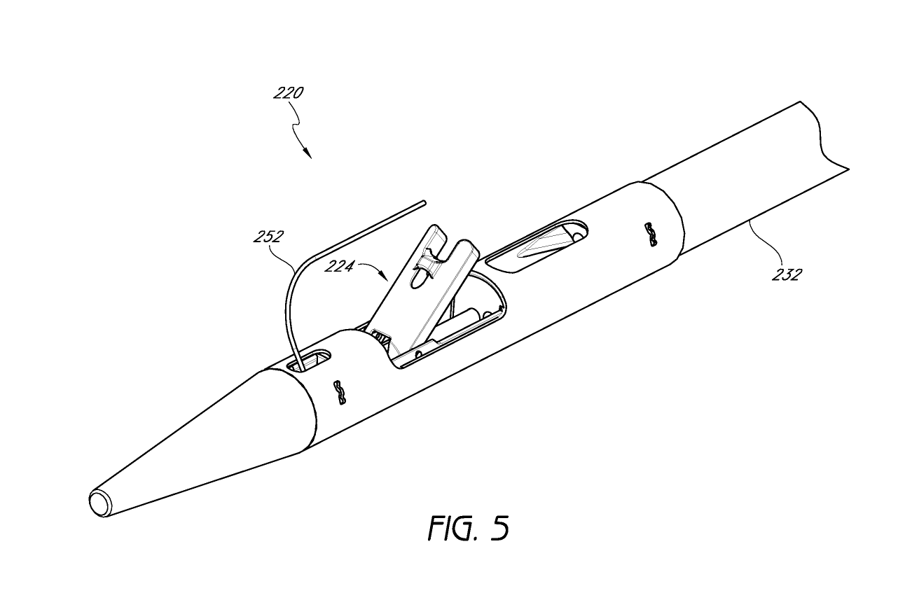

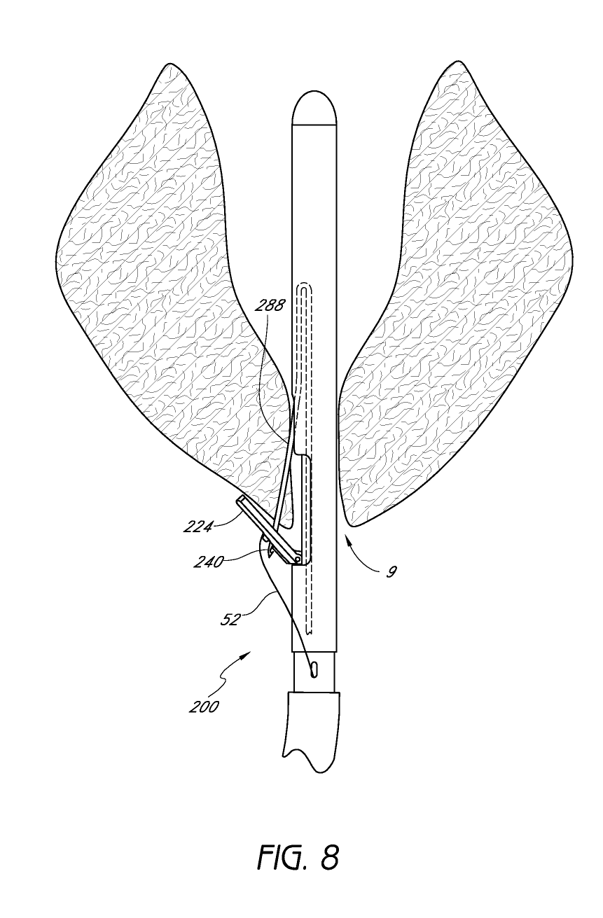

FIGS. 4A-4C and 5 illustrate other suturing devices 200 that can be used to place suture through heart tissue such as a transapical opening. Suturing device 200 can include a distal end 220 that can be operated using the handle portion described above. As shown, the suturing device 200 can comprise the distal end 220 with a tapered or rounded distal tip coupled to an elongate body 232, a handle 201, and an arm 224. In some embodiments, the elongate body 232 can have a constant diameter. In some embodiments, the device 200 can include two, four, six, eight arms or more, or alternatively, an odd number of arms. The distal end can further include features illustrated in the embodiments of FIGS. 3A-3C, such as a suture clasp 256, a suture arm aperture 250, a curved or slanted needle guide 248, a needle aperture 230, a distal tip 254, a hole 246, a suture 252 and an actuating rod 258. The distal end 220 can include a pin 222 that removably and pivotably couples a proximal portion of the arm 224 to an internal portion of the distal end 220. The pin 222 can be generally oriented perpendicular to the longitudinal axis of the distal end 220. A stop 226 can provide a positive stopping mechanism to prevent rotation of the arm 224 about pin 222 beyond a desired rotation angle. The distal end 220 can further include a needle 240 (FIG. 8). When the suture arm 224 is retracted into the suture arm aperture 250 and the needle 240 is retracted into the needle aperture 230, the arm 224 and the needle 240 can be recessed within the distal end 220. This can prevent the arm 224 and the needle 240 from causing tissue damage while the distal end 220 passes through a biological structure, such as the transapical opening in a human heart.

FIGS. 4B and 4C illustrate the distal end portion 220 of the suturing device 200 (FIG. 4A) with the suture arm 224 fully deployed distally and outwardly from the recessed position. Such deployment can be achieved by depressing an arm trigger 204, as described above with reference to FIGS. 2A through 2D. The actuating rod 258 can be coupled to the arm 224 at an offset distance from the pin 222, at the arm proximal portion 228. The offset distance provides a sufficient moment arm length to allow the actuating rod 258 to initiate a distal and radially outward movement of the arm 224. Depressing the arm trigger 104 translates the first follower member 126 and actuating rod 258 proximally, which pivots the suture arm 224 radially outward about the coupling to the pin 222. The arm 224, when deployed, can extend distally and can be inclined relative to the longitudinal axis of the device. As the arm trigger 104 is depressed further, the actuating rod 258 forces the suture arm 224 into a fully deployed state. The fully deployed state can be defined by the rotation terminating contact that occurs between the arm 224 and the stop 226. The suture arm 224 extends a distance of about 1 mm to 15 mm away from the outer diameter of the elongate body 232. In one embodiment, the suture arm 224 continues to deploy radially until the arm 224 is substantially at 45° to the longitudinal axis of the suturing device 100, as shown in FIGS. 4B through 4C. In some embodiments, the arm extends outward toward the distal end of the device 200 at an angle of between about 20-70°, 40-60°, 35-55°, alternatively about 40-50°, alternatively about 45° with respect to the longitudinal axis of the elongate body 232. In other embodiments, the suture arm 224 can be “fully” deployed when the arm 224 reaches an acute angle with respect to the longitudinal axis of the distal end 220, or an obtuse angle relative to each other.

The needle 240 (FIG. 4C) can be movable between a retracted position within the constant diameter elongate body 232 and the distal end 220 and an extended position that can engage arm 224, as illustrated, for example, in FIG. 8. As shown in FIG. 4C, some embodiments of the device 200 can have a needle 240 that can include a shaped distal end that bends back on itself 180 degrees, wherein the tip of needle 240 can point proximally. The needle 240 can extend proximally and radially outwardly from the elongate body in a distal-to-proximal direction to engage a suture 252 portion held by the arm 224.

FIG. 5 illustrates a second version of the device 200 where the arm when deployed extends proximally and can be inclined relative to the longitudinal axis of the device, with a needle that moves outwardly from the elongate body in a proximal-to-distal direction to engage a suture portion held by the arm.

Further details regarding these suturing devices 100, 200 and other devices, structures, and methods for suturing are shown and described in U.S. Pat. No. 7,090,686, U.S. Patent Application Publication No. 2008/0269786, published on Oct. 30, 2008, U.S. Patent Application Publication No. 2006/0069397, published on Mar. 30, 2006, and International Patent Application Publication WO 2009/137766, published on Nov. 12, 2009. The entireties of U.S. Pat. No. 7,090,686, U.S. Patent Application Publication No. 2008/0269786, and International Patent Application Publication WO 2009/137766 are incorporated by reference herein and form a part of this specification. In some embodiments, suture can be placed using a SuperStitch or NobleStitch device, available from Nobles Medical Technology, Inc. (Fountain Valley, Calif.).

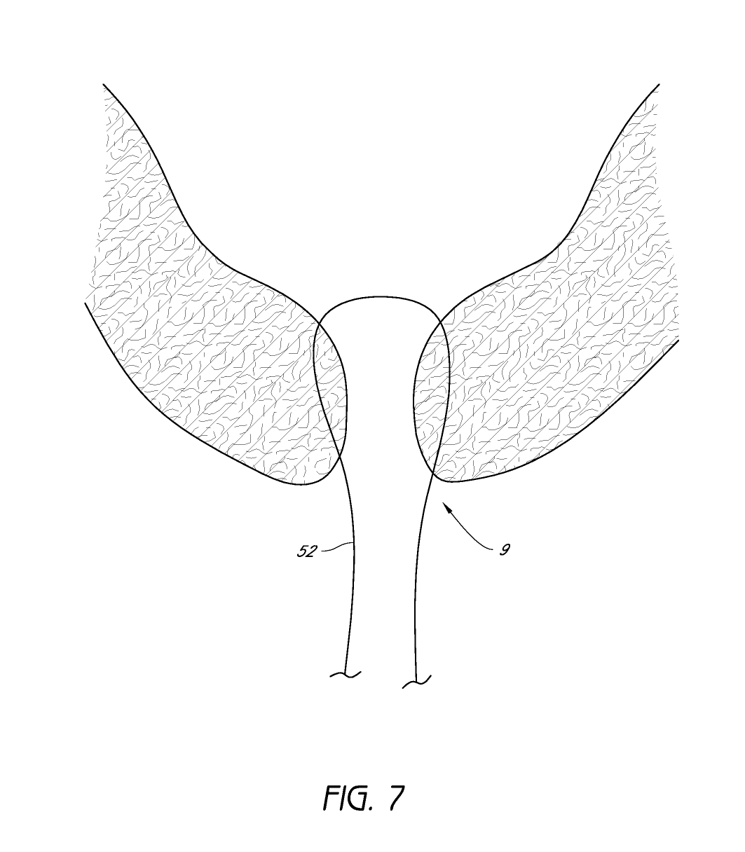

FIGS. 6-7 illustrate an embodiment of a method of closing a transapical opening using the suturing device 100 of FIGS. 2-3. The suturing device 100 can be introduced through the opening 9. As indicated above, the device 100 can be introduced into the opening with or without the aid of a guidewire 10 that may pass through a lumen of the elongate body 32 and the distal end 20 (FIG. 1). The device 100 can be positioned through the opening a sufficient distance to permit the arms 24 to be deployed (FIG. 6B) without damage to the surrounding tissue, as shown in FIG. 6A. With the arms 24 deployed, the device 100 can be retracted to cause the arms 24 to engage the heart internal wall tissue surrounding the opening 9, as illustrated in FIG. 6C. The needles 40 can be advanced through the heart external wall tissue and pass through heart tissue adjacent the opening 9, as shown in FIG. 6C, to snatch the end portions of the suture 52 from the arms 24. This step may be performed while maintaining a proximal retraction force to hold the arms against the internal wall tissue. The suture 52 can then be withdrawn through the heart tissue as the needles 40 are retracted into the elongate body 32, as illustrated in FIG. 7. The arms 24 can then be retracted and the entire suturing device 100 can be withdrawn. After the suture 52 has been placed the suture 52 ends can be tied together to close the opening 9.

Although the device 100 can be used for suturing transapical openings of the heart, the device 9 can be used to suture other tissues such as, by way of example, a patent ductus arteriosus, a patent foramen ovale (PFO), a heart defect, a puncture wound, and the like.

Before the procedure, the suture arms 24, 24′ can be pre-loaded with the ends of the suture loops 41, such as, for example, a polypropylene suture 52. Specifically, each end of a suture 52 can have a capture portion comprised of a loop 41, or a sphere or a ferrule. In one embodiment, the loop 41 can be formed (e.g., by heat molding) with the same suture material as the length of suture. In another embodiment, the loop 41 can be a separate piece attached (e.g., molded, glued, etc.) onto each end of the length of suture. The loop 41 can be loaded in respective clasps 56 of the arms 24, 24′. The remaining length of the suture extends through the constant diameter elongate body 32. With the sheath 12 extending through the patient's outer heart wall if a sheath 12 is used, the physician then inserts the distal end 20 through the sheath 12 and into the heart. The sheath 12 would be partially withdrawn along the elongate body 32 to remove the sheath 12 from the heart and to expose the needle apertures 30, 30′, as shown in FIGS. 3A-3C. The markers 68 on the exterior surface of the elongate body 32 can indicate how far the physician should withdraw the sheath 12 to expose the needle apertures 30, 30′.

The distal tip 54 of the distal end 20 can have a smooth, tapered or rounded surface tip which prevents injury to the opposite and adjacent internal heart walls when the distal end 20 is inserted into the transapical opening. In addition, blood flow within the heart can be uninterrupted because the distal end 20 does not occlude the heart blood flow. The physician can use bleed back through the hole 46 and the lumen 62′ (FIG. 3C) to determine when the distal end 20 has entered into the heart.

During insertion into the heart, the arm trigger 104 and needle trigger 106 are each in the non-depressed positions. As a result, the first follower 126 can be located in the distal position such that the suture arms are in the retracted condition. Also, the second follower 132 can be in the proximal position such that the needles are in the retracted condition.

While the distal end 20 is inserted into the heart, as shown in FIG. 6A, the actuating rod 58 holds the suture arms 24, 24′ in a recessed state within the distal end 20. The actuating rod 58 applies a downward proximal force while a pair of deflection surfaces 67 (FIG. 6A) adjacent the arm apertures 50, 50′ of the distal end 20 apply an inward force on each of the suture arms 24, 24′, respectively. The combination of these two forces retains the suture arms 24, 24′ within the suture arm apertures 50, 50′ of the distal end 20. Each of the suture clasps 56, 56′ can include an annular recess 80 which holds the suture 52 loop 41 as illustrated in FIGS. 11A through 11C. The loops 41 of the suture 52 are held securely by the suture clasps 56, 56′, but can be positioned for easy removal by a pair of hooks 42 at the tips of the needles 40, 40′.

Once the distal end 20 of the device 100 is properly positioned within the heart, the physician can depress the arm trigger 104 to deploy the suture arms 24, 24′ as shown partially deployed in FIG. 6B. Downward movement of the arm trigger acts on the first follower member 126 in the main housing 102, thereby causing the first follower member 126 to translate proximally, which pulls the actuating rod 58 proximally. The corner portion of the arm trigger 104 provides a camming surface which engages the inclined surface 128 on the first follower member 126. During this action, the force applied on the arm trigger 104 can be sufficient to overcome the biasing force of the arm spring 130. Movement of the first follower member 126 translates the actuating rod 58 proximally, which relieves the downward force applied by the actuating rod 58 and thus also relieves the inward forces applied to the suture arms 24, 24′ by the deflection surfaces 67. This allows the suture arms 24, 24′ to assume a partially deployed state as illustrated in FIG. 6B. As the physician continues depressing the arm trigger 104, the actuating rod 58 continues translating proximally, bringing the suture arms 24, 24′ into contact with the proximal inside edges 78, 78′ (FIG. 6A). The proximal inside edges 78, 78′ apply a downward force on each of the suture arms 24, 24′, respectively, thereby forcing the suture arms 24, 24′ into a fully deployed state, illustrated in FIG. 6C.

As the arm trigger 104 becomes fully depressed, the protrusion along the corner portion of the arm trigger 104 advances beneath the first follower body 126. In this position, the arm trigger 104 can be maintained in the fully depressed position by the force of the arm spring 130, which pushes the first follower member 126 against the arm trigger. Accordingly, the cooperation between the arm trigger and the first follower member 126 advantageously provides a releasable detent mechanism for holding the arm trigger 104 in the depressed position. When the arm trigger 104 is held in the fully depressed condition, the suture arms 24, 24′ are locked in the fully deployed condition.

In the locked state, the suture arms 24, 24′ have reached a fully extended position and can be longitudinally aligned with each other, as illustrated in FIG. 6C. With the suture arms 24, 24′ in this fully extended position, the physician can gently slide the suturing device 100 proximally so that the suture arms 24, 24′ contact the interior surface of the heart wall.

The physician then depresses the needle trigger 106 on the handle portion 101 to distally advance the needles 40, 40′ and capture the ends of the suture 52 from the suture clasps 56, 56′. FIG. 2A illustrates the needle trigger 106 in the non-depressed position. During downward depression of the needle trigger 106, the camming surface along the corner portion of the needle trigger 106 can engage and slide along the inclined surface 134 of the second follower member 132, thereby causing the second follower member 132 to slide longitudinally within the main housing 102 in a distal direction. During this action, the force applied on the needle trigger 106 can be sufficient to overcome the biasing force of the needle biasing spring 136. As the needle trigger 106 is depressed further, the second follower member 132 continues to slide distally, thereby advancing the needles distally through the main housing and through the elongate body 32. As the first and second needles 40, 40′ advance distally, the distal ends of the needles extend outward for engagement with the arms.

The paths taken by the needles 40, 40′ are illustrated in FIG. 6C. The needles 40, 40′ slide along the needle lumens 60, 60′ (FIG. 3C) and out of the suture device 100 through the needle apertures 30, 30′, respectively. When the needles 40, 40′ come in contact with the needle guides 48, 48′ (FIG. 3C), the needles 40, 40′ begin to bend radially outward. As the needles 40, 40′ exit, they can be guided at a radially outward, acute angle away from the actuating rod 58 by the needle guides 48, 48′. The angle of the needle deflection can be about 13.2 degrees. Deflection angles between about 10 degrees and about 15 degrees and between about 5 degrees and about 20 degrees can also be contemplated.

During advancement, the needles 40, 40′ penetrate the outer heart wall at an angle, thereby creating the needle incisions 88, 88′ on opposing sides of the opening 9. As mentioned above, the needles 40, 40′ also bend slightly (radially outward) when they come in contact with the suture arms 24, 24′. The annular recesses 80, 80′ of the suture clasps 56, 56′ and the hooks 42 can exert a force on each of the loops 41 of the suture 52 such that the loops 41 remain tied to the suture clasps 56, 56′ while the needles 40, 40′ pass therein.

The physician depresses the needle trigger 106 until the hooks 42 of the needles 40, 40′ engage the suture clasps 56, 56′ and capture the looped ends of the suture 52. As shown in FIG. 6C, the suture arms 24, 24′ hold the looped ends of the suture 52 away from the distal end 20 so that the needles 40, 40′ pierce the outer heart wall and capture the loops 41 of the suture 52 outside the perimeter of the distal end 20. Mechanical limits prevent additional movement of the needle trigger 106 once the needles 40, 40′ have optimally engaged the suture clasps 56, 56′. Such resistance signals to the physician that the needles 40, 40′ have reached an optimal, predetermined location within the suture clasps 56, 56′.

In some embodiments, a needle trigger 106 can include first and second pins 144, 146 and a gap therebetween (FIG. 2D). The needle trigger is configured for cooperation with the follower member 132 (FIG. 2D). In one embodiment, the pins 144, 146 of the needle trigger 106 initially ride along the inclined surface 134 of the follower member 132, thereby causing the follower member to move in a distal direction for extending the needles. However, as the needle trigger 106 reaches a finish (i.e., fully depressed) position, the pins 144, 146 extend beyond the bottom edge of the inclined surface, thereby relieving the force on the follower member and allowing the follower member to snap back in a proximal direction. This occurs while maintaining the needle trigger 106 in the fully depressed condition. Accordingly, the needles are first fully extended and then automatically snap back when the needle trigger reaches a first finished position (i.e., is fully depressed). The needles snap back due to the spring biasing of the follower member 132. When the needle trigger is released, the pins ride back up via slots (not shown) to the start position. Spring mechanisms (not shown) can bias the arm and needle triggers 104, 106 back into the start position to facilitate the automatic retraction of the trigger while with the needle biasing spring 136 can simultaneously urge the follower member 132 proximally.

In some embodiments, the relationship between the needle trigger 106 and the follower member 132 can be configured such that the needles retract from the first finished position at a first rate and then retract from a second finished position at a faster rate. This may be achieved by providing a cut away portion (not shown) on the follower member. This retraction of the needles at a slow rate followed by a fast rate advantageously provides a “pre-tensioning” of the suture such that the needles initially tug slowly on the suture ends and then more quickly. The initial slow tugging allows the suture ends to become better aligned before withdrawal through the tissue.

In some embodiments, the needle trigger does not automatically retract. After the physician advances the needles 40, 40′ to the predetermined location within the suture clasps 56, 56′, the physician releases pressure on the needle trigger 106, thereby allowing the needle biasing spring 136 within the handle portion 101 (see FIGS. 2A-2D) to retract the needles 40, 40′ proximally. This motion causes the needles 40, 40′ to withdraw into the needle lumens 60, 60′ with the loops 41 of the suture 52 attached to the hooks 42. The hooks 42 capture the looped ends of the suture 52 held by the suture clasps 56, 56′ and pull the loops 41 up through the needle incisions 88, 88′ as the needles 40, 40′ retract proximally. As the needles 40, 40′ pull proximally on the loops 41 of the suture 52, tension in the suture 52 causes additional segments of the suture 52 to feed through the hole 46 at the distal tip 54 of the distal end 20, into the heart and through the needle incisions 88, 88′. In this embodiment, the physician can regulate the rate of needle movement by controlling the rate of movement of the needle trigger. From the above, it can be seen that the position of the needles can be substantially directly proportional with the position of the needle trigger. Accordingly, by sensing the position of the needle trigger, the physician can be provided with a reliable indication of needle position at any given time.

In the above-described embodiment, the physician advantageously controls the position of the needles 40, 40′ by depressing and releasing the needle trigger 104. The advancement of the needle can be achieved by depressing the needle trigger in a controlled manner, while retraction can be achieved by allowing the needle spring to retract the needle while the physician regulates the rate of retraction with the needle trigger. Once the needles 40, 40′ have been retracted into the needle lumens 60, 60′, the physician depresses the arm release button 108 to release the arm trigger 104. The arm release button urges the corner portion of the arm trigger 104 in a distal direction such that the protrusion can be released from the first follower member 126, thereby allowing the arm trigger 104 to spring back upward.

Once the arm trigger 104 is released, the arm biasing spring 130 can push the first follower member 126 distally, thereby moving the actuating rod 58 distally. This relieves the forces applied to the suture arms 24, 24′ by the proximal inside edges 78, 78′ (FIG. 6A), allowing the suture arms 24, 24′ to assume a relaxed state as illustrated in FIG. 6B. Upon further distal movement of the first follower member 126, the suture arms 24, 24′ move distally until contacting the deflection surfaces 67, 67′ (FIG. 6A). Together with the deflection surfaces 67, 67′, the downward force of the actuating rod 58 causes the suture arms 24, 24′ to retract into the recessed state within the distal end 20, as shown in FIG. 6A. In the recessed state, the suture arms 24, 24′ can be substantially parallel with the elongate body 32, and the exterior surfaces of the suture arms 24, 24′ can be substantially flush with the exterior surface of the distal end 20. This reduces the likelihood that the suture arms 24, 24′ will snag or catch on the heart outer wall or other body tissue during withdrawal. With the suture arms 24, 24′ and the needles 40, 40′ returned to the recessed state, the device 100 can be ready for removal from the heart.

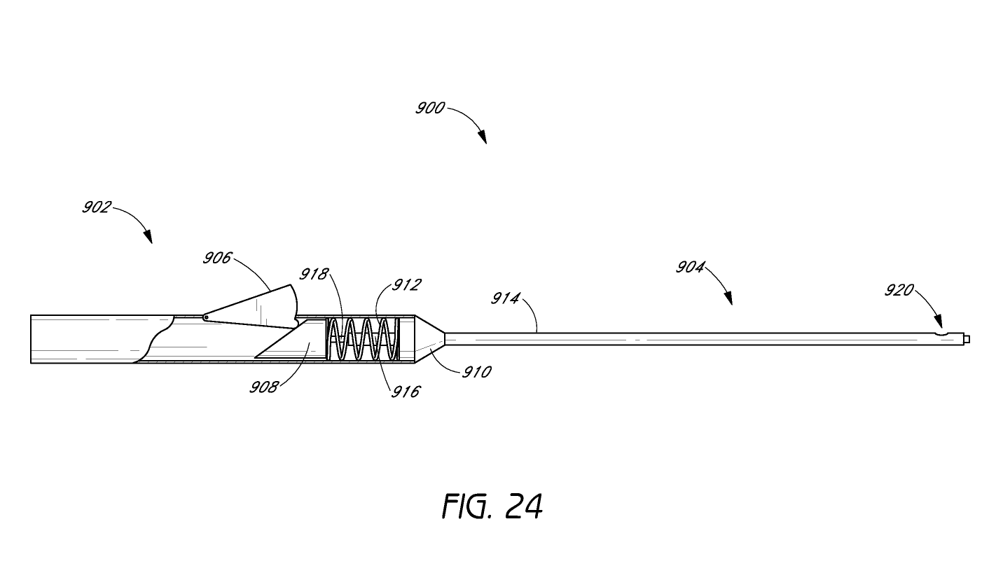

The physician then withdraws the device 100 out of the heart outer wall transapical opening and out of the patient's body. After the device 100 is fully withdrawn (and possibly with the sheath 12, if used, still in the body), the physician gently pulls the ends of the suture 52 and may apply a knot to close the transapical opening 9, see FIG. 7. In the embodiment wherein the suture 52 passes through the needle incisions 88, 88′, when the ends of the suture 52 are pulled, tension in the suture 52 closes the transapical opening 9. The physician then ties at least one knot with the ends of the suture 52 and slides or pushes knot(s) down through the sheath 12 to the transapical opening 9. The physician can tie and push the knot(s) by using any suitable suture knot tying and/or cinching apparatus. The physician can tie the sutures 52 via applying a knot device as described below and illustrated in FIGS. 24-26. Alternatively, the physician can tie at least one knot by hand and then cinch the knot by using a knot cinching device. Still, the physician can choose to fasten a small, circular or flat stainless steel clip (not shown) to the ends of the suture 52 and slide the clip down through the sheath 12 to the transapical opening 9 to close the opening 9. The physician then cuts the unused ends (extra length) of the suture 52 and removes the cut portions. The physician then removes the sheath 12 from the patient's body.

With the suture end portions extending from the heart, the suture can be secured to close the opening. In some embodiments, a knot can be tied according to any known method or by applying a knot, such as described in U.S. Patent Publication No. 2007/0010829 A1, published Jan. 11, 2007, the entirety of which is incorporated by reference herein and forms a part of this specification.

In some embodiments, the suturing apparatus can be provided with a lumen for slidably receiving a guidewire. In one example, the guidewire lumen can be combined with the bleed back lumen. The guidewire lumen can assist the physician during insertion of the suturing apparatus into the patient and advancing the device toward the treatment site.

FIGS. 8-10 illustrate an embodiment of a method of closing a transapical opening using the suturing device 200 of FIGS. 4A-4C. This method of closing is similar to the method described above for FIGS. 6-7. The primary differences for FIGS. 8-10 can include the distally directed extended arm at an acute angle to the distal end longitudinal axis, the proximally extending needle, and the placement of the extended arms adjacent the outer surface of the heart outer wall, rather than internal to the heart. A first suturing device 200 can be introduced through the opening 9, with or without the use of a guidewire 10 (FIG. 1). The arm 224 can be deployed and the device 200 can be advanced through the opening 9 until the arm 224 engages the tissue near the opening 9, as illustrated in FIG. 8, along an outer surface of the heart outer wall. Alternatively, the device 200 can be advanced through opening 9 with arm 224 retracted, and after advancement, the device can be pulled back proximally and the arm can be deployed. In some embodiments, the arm 224 has a fully deployed configuration that can be approximately parallel to the outer surface of the heart to which it is intended to be engaged. With the arm 224 advanced distally against or adjacent to the outer surface of the heart, the needle 240 can be advanced through the tissue at a needle incision 288, can catch the suture 52, and can be retracted through the tissue with the suture end portion 52. The suturing device 200 can be removed from the opening 9 while leaving the suture 52 extending through the tissue.

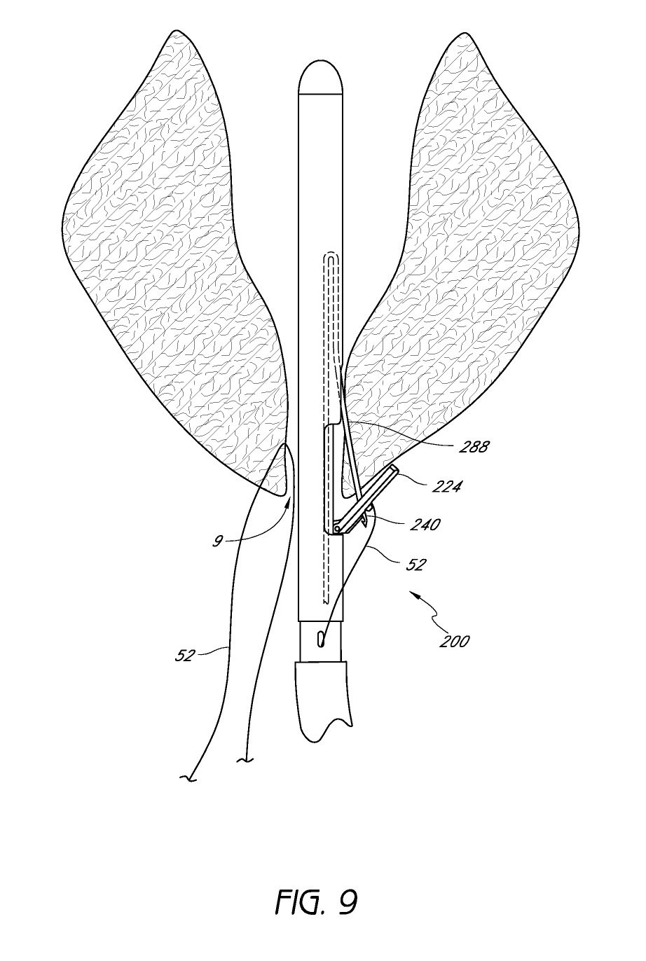

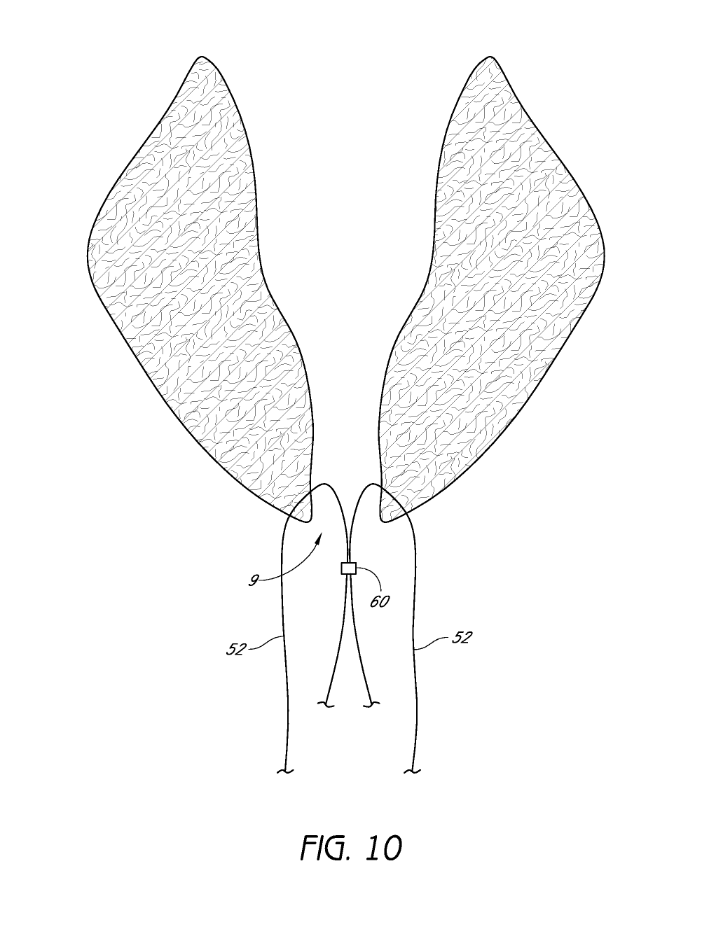

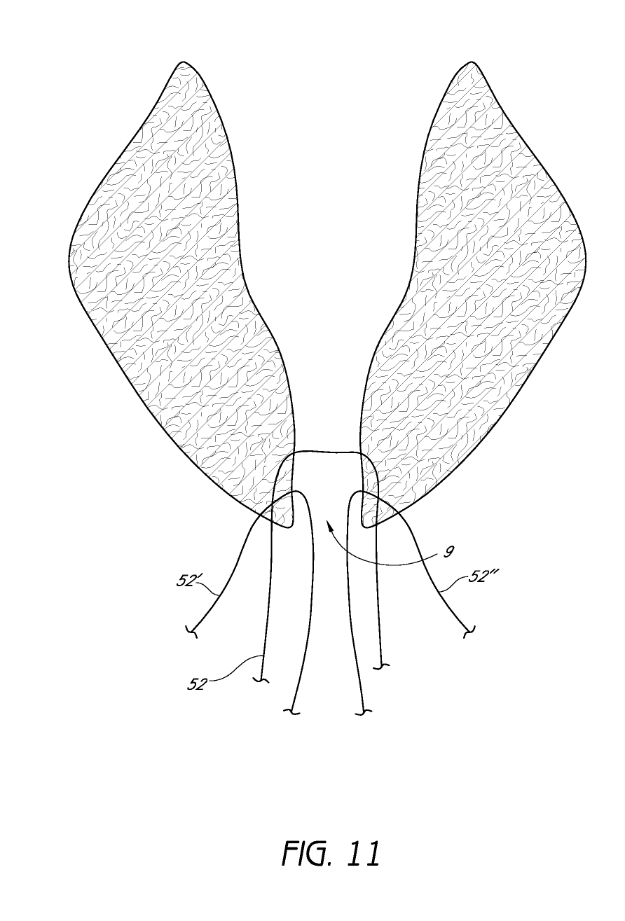

With reference to FIG. 9, a second suturing device 200 (or the same suturing device 200) can be introduced into the opening 9, with or without the aid of a guidewire 10 (FIG. 1). The second suturing device 200 can be oriented such that the needle 240 can be placed generally opposite the first suture across the opening 9. For example, the needle 240 and arm 224 of the second suturing device 200 can be oriented about 180 degrees rotated from the orientation of the needle 240 and arm 224 of the first suturing device 200. A second suture can be placed through the tissue following a procedure similar to that described above in connection with the first suturing device 200. The second suturing device 200 can be withdrawn from the opening 9, leaving two sutures extending through opposing tissue portions, as illustrated in FIG. 10.

The two sutures 52 can be joined together, for example by tying or otherwise applying a knot 60 to the portions of the sutures which extend through the opening 9, as shown in FIG. 10, for example. Thereafter, the suture portions extending through tissue near the opening 9 can be tightened and secured together as described above to close the opening 9.

In some embodiments, the suturing device 100 of FIGS. 2 and 3 and the suturing device 200 of FIGS. 4A-4C can be used together to close an opening. A suture can be placed by the device 100, as described above in connection with FIGS. 6 and 7. Two additional sutures can be placed using two devices 200, as described above in connection with FIGS. 8-10. These sutures can be placed in any order. In one embodiment, the earlier-placed sutures can be held aside while later sutures are placed. FIG. 11 illustrates a transapical opening 9 with a suture 52 having been placed by the suturing device 100 and sutures 52′, 52″ each having been placed by a corresponding suturing device 200. In a preferred embodiment the suturing device 100 of FIGS. 2 and 3 places the first suture and suturing devices 200 of FIGS. 4A-4C place second and third sutures. In some embodiments, however, other placement orders can be used; for example, one or more suturing devices 200 can be used to place sutures before the suturing device 100. The sutures can be secured together in any manner, including those discussed above, to close the opening 9.

In some embodiments, a single suturing device having forwardly-extending (or distally-extending) needles 240 and rearwardly-extending (or proximally-extending) needles 240 can be used to close an opening. For example, in some embodiments a suturing device can have two forwardly-extending needles 240 and two rearwardly-extending needles 240, and arms configured to hold sutures 52 for retrieval by the needles 240 when the arms 224 are in a deployed position. In some such embodiments, all of the needles 240 can be actuated simultaneously, while in other embodiments some needles can be actuated before others. For example, the forwardly-extending needles 240 might be actuated before the rearwardly-facing needles 240. In some embodiments, the suturing device can comprise more than four needles 240, for example, 5, 6, 7, or 8 needles 240.

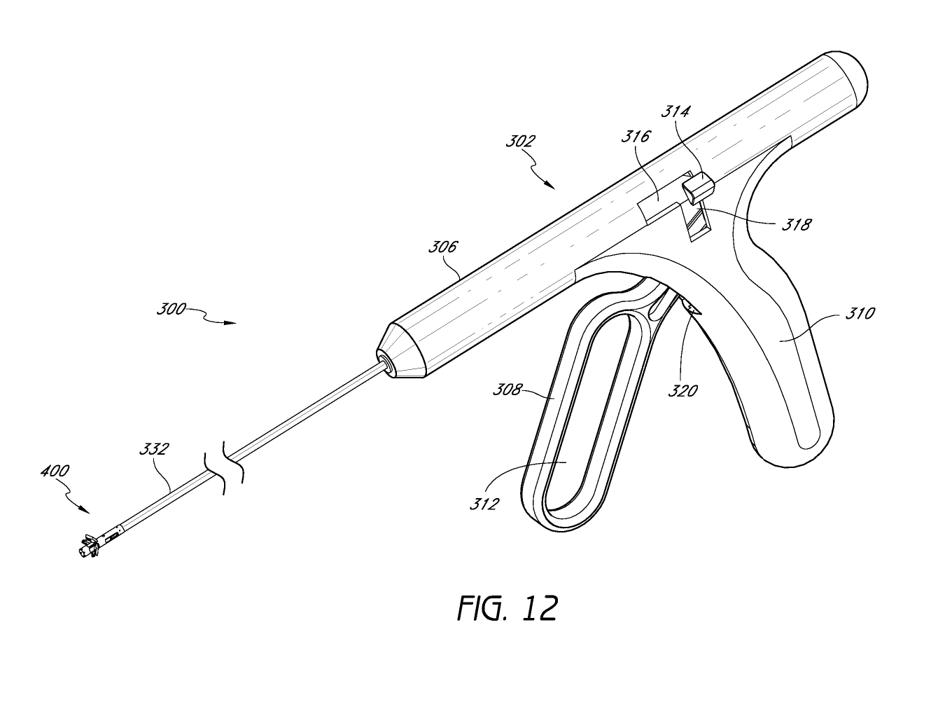

FIG. 12 illustrates a suturing device 300 in accordance with another embodiment. The apparatus can include, generally, an elongate body 332 for insertion into an internal biological structure, e.g. accessing the human heart, and a handle portion 302. In some embodiments, the elongate body 332 can have a constant diameter. The handle portion 302 functions in a similar manner as the embodiment shown in FIGS. 2A-2D, utilizing a trigger-handle type of geometry. The handle portion 302 can include a main body 306, a trigger actuator 308 for actuating a needle 440, a lever actuator 314 for the suturing mechanism arm 424, and a handle 310 for gripping and manipulating the device 300. The elongate body 332 can be flexible to allow it to bend when advanced through an internal biological structure, such as when accessing the heart. The length of the elongate body 332 can be modified to accommodate various suturing applications, and in some embodiments, has a length of about 15 to 80 cm, more preferably less than about 80 cm, 70 cm, 60 cm, 50 cm, 40 cm or 30 cm. The trigger 308 can be formed with a finger aperture 312 to ensure secure engagement with the physician's hand. A lever 314 can be provided for controlling the deployment of the distal suturing components and can be contained within a horizontal slot 316 and a vertical slot 318 on the handle main body 306. The trigger 308 and lever 314 can be operatively connected to the distal portion 400 of the suturing device 300 and can be used to remotely manipulate the components of the distal portion 400.

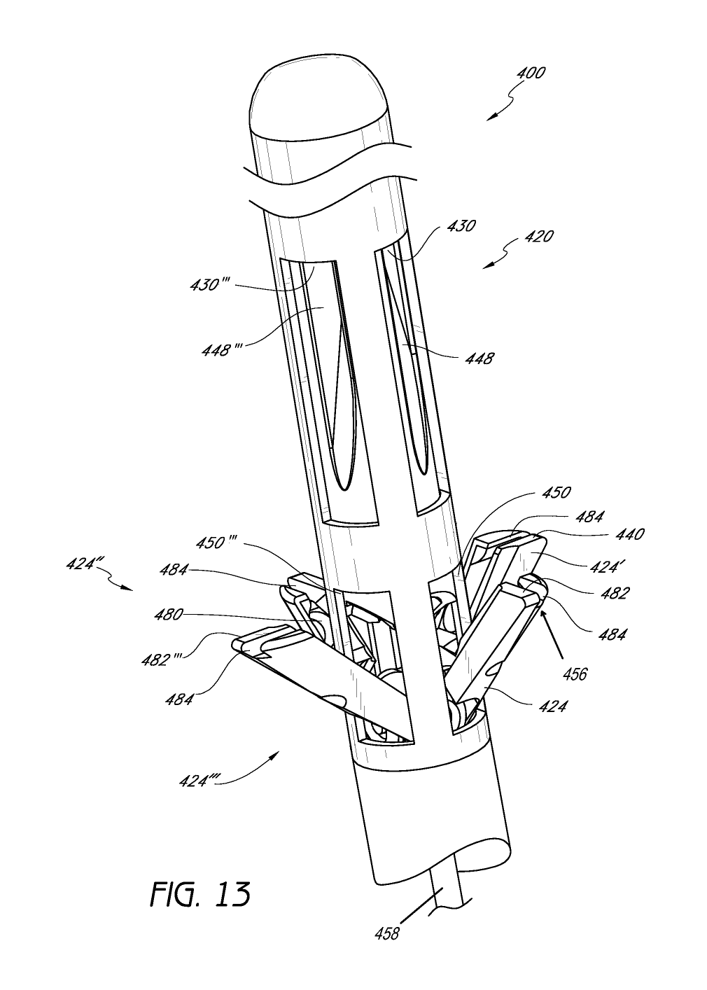

FIG. 13 illustrates the distal portion 400 of the device 300 in greater detail. The distal portion 400 can be similar to the distal portion 220 illustrated in FIGS. 4A-4C, having four symmetrical arms 424 located at approximately or substantially 90 degrees to each other. In some embodiments, the device 400 can include two, four, six, eight arms or more, or alternatively, an odd number of arms. The distal portion 400 advantageously provides for placement of multiple sutures simultaneously in a manner similar to that described by FIGS. 4A-4C.

The distal portion 400 can include an elongate body 432 and a distal end 420 that can include a plurality of arms 424, 424′, 424″, 424′″, a plurality of arm apertures 450, 450′, 450″, 450′″, a plurality of needle apertures 430, 430′, 430″, 430′″, a plurality of needles 440, 440′, 440″, 440′″, a plurality of curved needle guides 448, 448′, 448″, 448′″, and an actuating rod 458. In some embodiments, the elongate body 432 can have a constant diameter. In some embodiments, the distal portion 400 can include four arms. When the arms 424-424′″ are retracted into the arm apertures 450-450′″, the arms can be recessed within the distal end 420 so that the arms do not cause tissue damage upon insertion and retraction of the distal portion 400 from a biological structure.

FIG. 13 illustrates details of an embodiment of the arm 424. The other arms 424′-424′″ can be generally identical to the arm 424 and symmetrically oriented about the distal end 420. The arm 424 can include a sloped end 482, an annular recess 480, a clasp 456, an annular recess 480, and a deflecting plate 486. The arms 424-424′″ can be individually actuated by the actuating rod 458, or via a hinged combination, or equivalent, of two or four of the arms combined, or of any other odd or even combination of arms thereof. The distal end of the actuating rod 458 (the end furthest from the handle 101 of FIG. 2A) can be attached to hinge portions of the arms 424-424′″ via a pin (not shown) similar to that depicted in FIG. 4C, for example. Actuation of the actuating rod 458 controls the movement of the arms 424-424′″.

Before operation, the arms 424-424′″ can be pre-loaded with the ends of one or more sutures 452, such as a polypropylene suture. The arms 424-424′″ can be preloaded with just one suture or up to four sutures. In some embodiments, more than four sutures 452 can be preloaded. Specifically, each end of a suture has a capture portion comprised of a loop 442, or a sphere or a ferrule. In one embodiment, the loop 442 can be formed (e.g., by heat molding) with the same suture material as the length of suture. In another embodiment, the loop 442 can be a separate piece attached (e.g., molded, glued, etc.) onto each end of the length of suture. The loop 442 can be loaded in each clasp 456 (FIG. 4) of the arms 424-424′″. The slit 484 receives a portion of the suture which adjoins the loop 442. The remaining length of the suture 452 can be loaded into the distal end 420 and into one of the lumens 448-448′″ shown in FIG. 13.

When the lever actuator 314 is moved upwardly, the actuating rod 458 can translate proximally. As the actuating rod 458 translates proximally, the sloped ends 482, 482′, 482″, 482′″ of the arms 424-424′″ pivot to deploy radially. The suture arms 424-424′″ extend a distance of about 1 mm to 15 mm away from the outer diameter of the elongate body 32. In one embodiment, the arms 424-424′″ continue to deploy radially until the arms 424-424′″ are substantially at 45 degrees to the longitudinal axis of the distal end 420. In other embodiments the arms 424-424′″ can deploy until they are substantially parallel to each other and perpendicular to the axis of the distal end 420, as shown in FIG. 13. In other embodiments, the arms 424-424′″ can be considered fully deployed when they reach an acute angle with respect to the longitudinal axis of the distal end 420, or an obtuse angle relative to each other. When the arms 424-424′″ are fully deployed, either parallel to each other or at an angle, the physician can squeeze the trigger actuator 308 to move the needles 440, 440′, 440″, 440′″ proximally. In one embodiment, the needles 440, 440′, 440″, 440′″ can be moved proximally at substantially the same time. In another embodiment, the needles 440, 440′, 440″, 440′″ can be actuated separately so that one needle 440, or pair of needles 440, 440′, moves before the other needles 440″, 440′″, or any combination thereof.

The needles 440-440′″ move proximally at an angle or along a curved path until the hooks 442 of the needles 440-440′″ engage the suture loops 441 of the suture 452 ends lying within the clasps 456, 456′, 456″, 456′″. Such engagement causes the suture loops 441 to become attached to the ends of the needles 440, 440′, 440″, 440′″, respectively, via the hooks 442. The physician then returns the trigger actuator to its original position to cause the needles, with the ends of the suture 452 attached to the ends of the needles, to retract proximally back into the distal end 420 and the constant diameter elongate body 332. In some embodiments, the needle trigger 106 retracts automatically, as described above. The physician then moves the lever 314 actuator such that the actuating rod 458 translates proximally. As the actuating rod 458 translates proximally, the arms 424-424′″ return to their retracted position. The physician then removes the distal portion 400 from the patient. As tension is applied to the suture 452 ends, the length of the suture 452 in the distal end 420 can be pulled out of the distal portion 400.

The suturing device 300 of FIG. 12 can be used to place suture 452 via a variety of access means to the heart, in particular transapical openings, as well as at other biological structures. In general, the physician inserts the distal portion 400 into the heart apex opening to place a suture 452 through the surrounding heart tissue portions. The distal portion 400 can be withdrawn from the patient to draw the two suture 452 ends outside of the patient. The physician ties a knot with the suture 452 ends, slides the knot down to the suture site, and cuts the lengths of suture 452 that are unused.

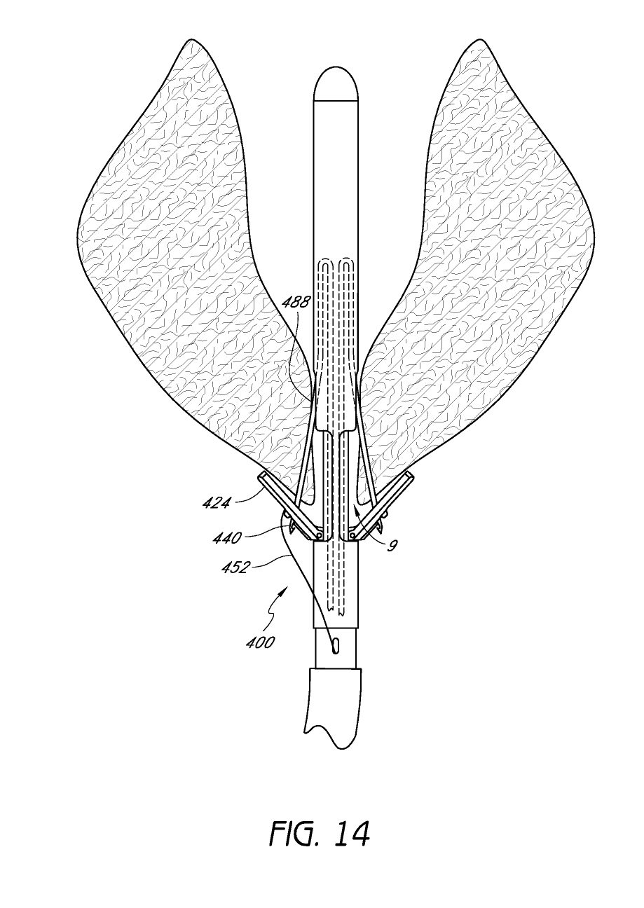

FIG. 14 illustrates an embodiment of a method of closing a transapical opening using the suturing device 300 of FIGS. 12 and 13. The method of using suturing device 300, and distal end 420 can be similar to the methods described in connection with FIGS. 6-11, with the exception that the plurality of sutures 452 can be placed with the single suturing device 300, generally simultaneously during a single device insertion. However, in other embodiments, a suture 452 can be placed one at a time. For example, device 400 can be introduced through the opening 9, with or without use of a guidewire 10. The arms 424-424′″ can have one or a plurality of sutures 52 coupled to the clasps 456-456′″. The arms 424-424′″ are deployed and the device 400 is advanced through the opening 9 until the arm 424-424′″ engage the heart tissue near the opening 9, similar to method illustrated in FIG. 8, along an outer surface of the heart. In one embodiment, the arms can have a deployed configuration that is approximately parallel to the outer surface of the heart to which it is engaged. The needles 440-440′″ are advanced through the heart tissue, catch the sutures 52, 52′, and are then retracted through the heart tissue with the loops 41 of suture end portions 52, 52′. The suturing device 400 is then removed from the opening 9 while leaving the sutures 52, 52′ extending through the heart tissue.

In another embodiment, a second suturing device 400 (or the same suturing device 400) can be introduced into the opening 9, with or without aid of a guidewire 10. The second suturing device 400 is oriented such that the needles 440-440′″ are placed generally opposite, or circumferentially offset from, the first suture sites around the opening 9. For example, the needles 440-440′″ and arm 424-424′″ of the second suturing device 400 can be oriented about 45 degrees rotated from the orientation of the needles 440-440′″ and arm 424-424″ of the first suturing device 400. A second set of sutures 52 can be placed through the heart tissue following a procedure similar to that described above in connection with the first suturing device 400. The second suturing device 400 is then withdrawn from the opening 9, leaving two sets of a plurality of sutures 52 extending through opposing heart tissue portions.

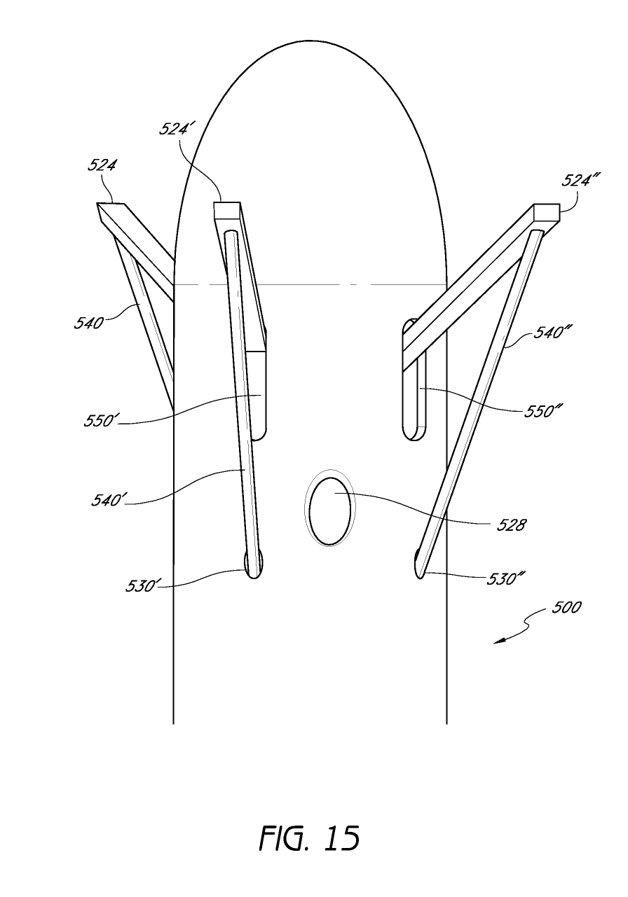

FIG. 15 illustrates an embodiment of a distal portion 500 that can be used in conjunction with the suturing device of FIG. 12, which illustrates another embodiment of a suturing device wherein multiple sutures can be applied simultaneously. The distal portion 500 of a suturing device can include four arms 524, 524′, 524″, 524′″ and four needles 540, 540′, 540″, 540′″ that can be equally spaced circumferentially, approximately or substantially about 90 degrees apart, about the distal portion 500 of the suturing device. In some embodiments, the device 500 can include two, four, six, eight arms or more, or alternatively, an odd number of arms. The needles 540-540′″ can be located proximally of the arms 524-524′″, such that the needles extend distally toward the distally extended arms 524-524′″, rather than the needles extending proximally, as illustrated in the embodiments of FIGS. 13-14. The distal portion 500 can simultaneously apply two perpendicular sutures 542 to the heart external wall biological structure. In some embodiments, the suturing device can be formed with any even number of arms and needles, such as, for example, six or eight. In some embodiments, the suturing device can be formed with an odd number of arms and needles, such as, for example, three or five. The distal portion 500 is described in greater detail below.

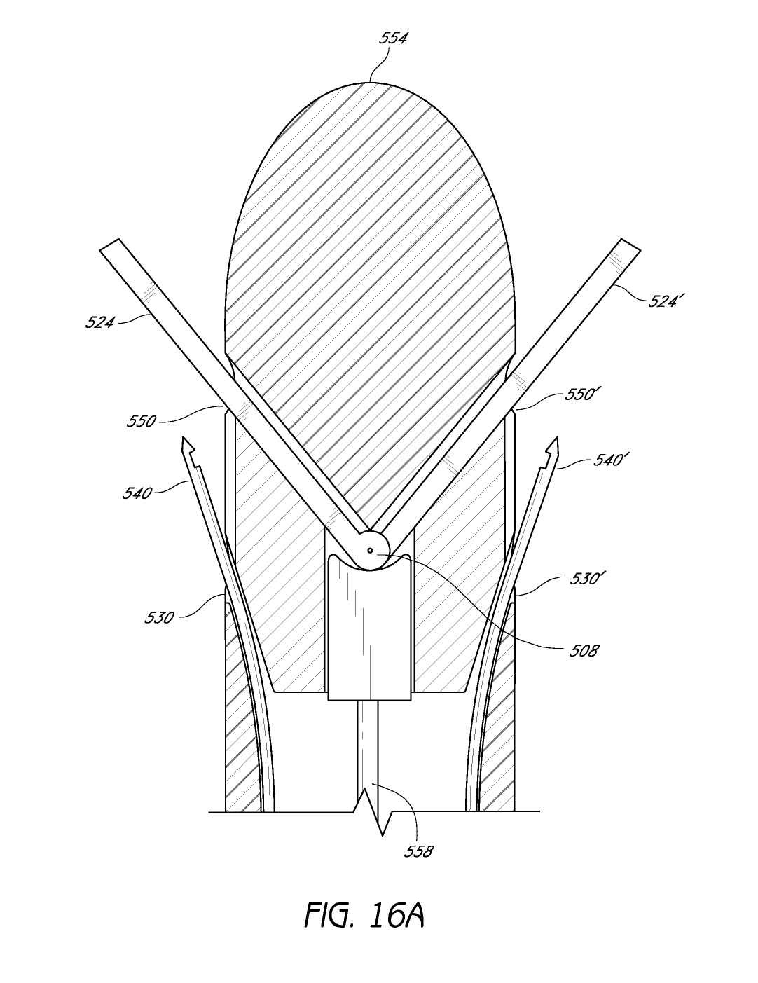

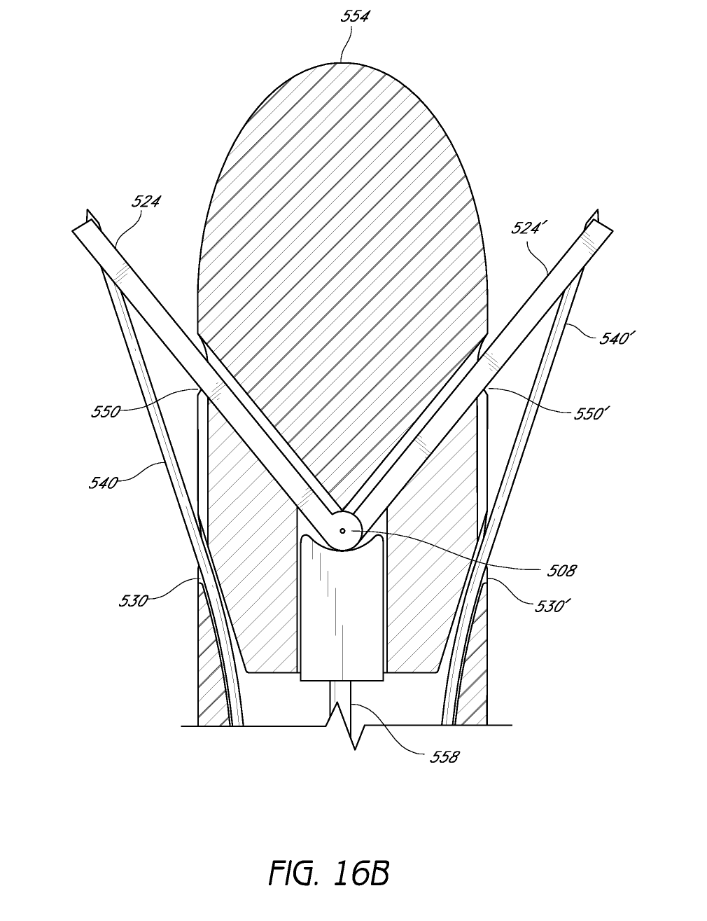

With reference to FIG. 16A, the distal portion 500 can include a tapered or smooth rounded distal tip 554, a plurality of arm apertures 550, 550′, 550″, 550′″, and a plurality of needle apertures 530, 530′, 530″, 530′″. The distal tip 554 can be adapted for insertion into a transapical opening in the heart or a similarly shaped structure and provides a means to enable access into narrow passageways or openings, generally facilitated by a smooth tapered, or rounded geometry. The distal tip 554 can be used to place the suturing device in optimum position of contact within the surrounding tissue. The arms 524-524′″ extend through the arm apertures 550-550′″ for penetrating the surrounding tissue of, for example, a biological structure such as the heart outer wall. The needles 540-540′″ extend through the needle apertures 530-530′″ for entering the heart tissue and capturing the loops 41 end portions of the sutures 52 from the arms 524-524′″, respectively, and withdrawing them back through the heart tissue and toward the device 500. An opening 528 can be provided adjacent to or on the distal tip 554 to provide a location for the suture 52 material to extend out of the device 500.

FIGS. 16A-16B illustrate the movement of the arms 524-524′″ and needles 540-540′″ of the suturing device distal portion 500 shown in FIG. 15. FIG. 16A shows the arms 524-524′″ in an extended position within the apertures 550-550′″ in the distal portion 500 of the suturing device. In the recessed position, the arms can be fully contained within the suturing device and can be configured in a substantially parallel arrangement. The proximal ends of the arms can be coupled together by a hinge 508. In FIG. 16A, the arms are shown extended such that the distal end of each arm contacts a spreader mechanism 512 thereby causing the arms 524-524′″ to separate. As the arms extend, they can be guided outward through the arm apertures 550-550′″. In FIG. 16B, the arms 524-524′″ are shown in the fully deployed position, such that each arm extends outward distally and radially away from the proximal end of the device. In FIG. 16B, the suturing device can be shown with the needles 540-540′″ and the arms 524-524′″ in the extended position, such that the distal end of the needles engage the clasps 556-556′″ of the arms 524-524′″.

In some embodiments, the four arms 524-524′″ can be configured to pivot distally and radially outward to extend away from the longitudinal axis of the distal portion 500, in a manner similar to the embodiment illustrated in FIGS. 12-14. The arms 524-524′″ can be individually actuated by an actuating rod 558, or via a hinged combination, or equivalent, of two or four of the arms combined, or of any other odd or even combination of arms thereof. The distal end of the actuating rod 558 can be attached to hinge portions of the arms 524-524′″ via a pin (not shown) similar to that depicted in FIG. 4C, for example. Actuation of the actuating rod 558 controls the movement of the arms 524-524′″. The arms 524-524′″ can be fully deployed to a distally pointing direction, when they reach an acute angle with respect to the longitudinal axis of the distal portion 500, or an obtuse angle relative to each other. The suture arms 524-524′″ extend a distance of about 1 mm to 15 mm away from the outer diameter of the elongate body 32. Upon the arms 524-524′″ being fully deployed, the needles 540-540′″ can be extended distally from the housed retracted position to the extended position to engage the suture and the arms 524-524′″.

In an alternative embodiment, the needles 540-540′″ can be located distally of the arms 524 on the distal end 520, similar to the embodiment shown in FIGS. 12-14. In such an embodiment, the needles 540 would extend proximally to engage the suture in the arms 524, thus reversing the direction that the sutures will be retracted through the heart tissue, as compared to the embodiment of FIGS. 15-16.

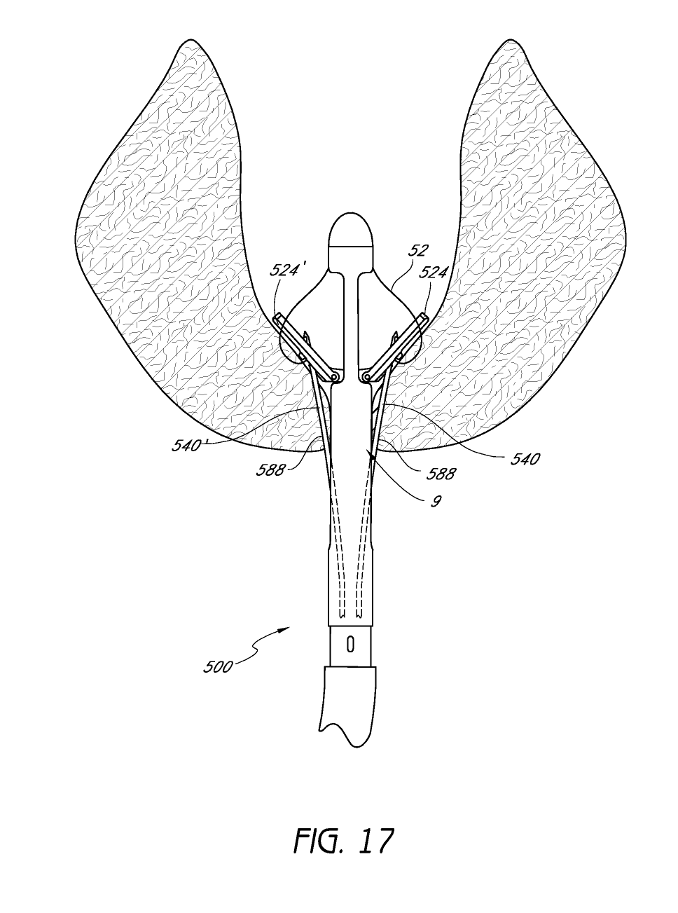

FIG. 17 illustrates an embodiment of a method of closing a transapical opening using the suturing device 500 of FIGS. 15-16. The steps involved can be similar to the methods described above in connection with FIGS. 12-14, with the exception of the direction of deployment of the needles 540-540′″. The device 500 can include arms 524-524′″ that extend distally after the distal end 520 is located within the internal cavity of the heart outer wall. The distal extension places the arms 524-524′″ adjacent or against the heart cavity internal wall surfaces. The needles 540 can then be distally extended through the heart external wall tissue, from the outside surface to the internal cavity, to contact the clasps 556-556′″ of the arms 524-524′″, and secure the loops 541 of the sutures. Retraction of the needles 540-540′″ then extracts the sutures 552 through the needle incisions 588 created in the heart wall tissue. Retraction of the arms 524-524′″ into the distal end 520 then allows removal of the suturing device 500 from the heart cavity and the body. The suture loops 541 remain secured to the needle hooks 542 to extract the suture 552 ends out of the body for subsequent knot securement and closing of the transapical opening 9.

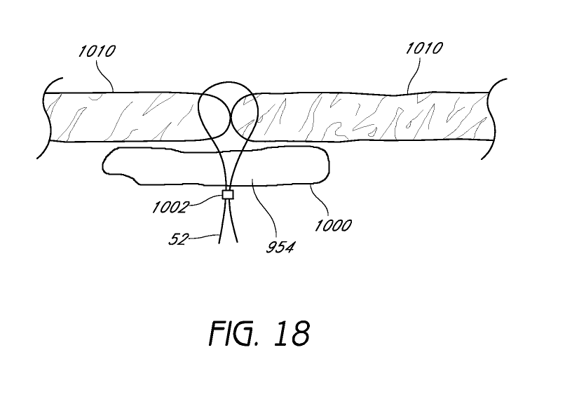

FIG. 18 illustrates a suture 52 placed adjacent an opening using any of the devices described above. FIG. 18 further illustrates placement of a pledget 1000 adjacent the outside surface of the tissue, for example the outside surface of the heart adjacent a sutured transapical opening, to absorb bodily fluid, e.g. blood, adjacent the opening. The pledget can be delivered over suture portions extending away from the opening, and as illustrated, may be deployed with a knot 1002 that secures the suture 52 in closing the transapical opening 9. In another embodiment, the knot 1002 can be placed adjacent the outside surface of the heart, and the pledget 1000 placed over the knot, to provide for readily accessible removal of the temporary placement of the pledget 1000 adjacent the opening 9. Placement of multiple sutures 52 at the opening 9 can provide increased securement of the pledget adjacent the opening 9. In another embodiment, a knot 1002 can be applied both at the heart outer surface wall and on the outer surface of the pledget 1000.

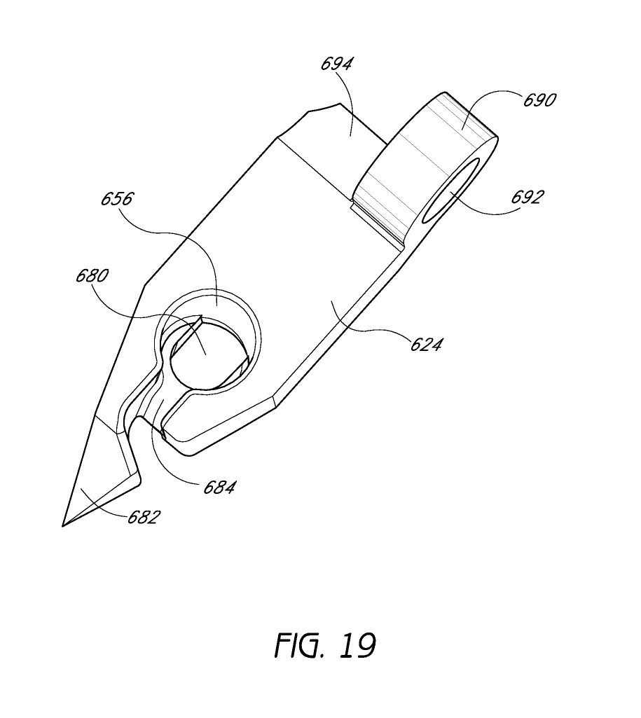

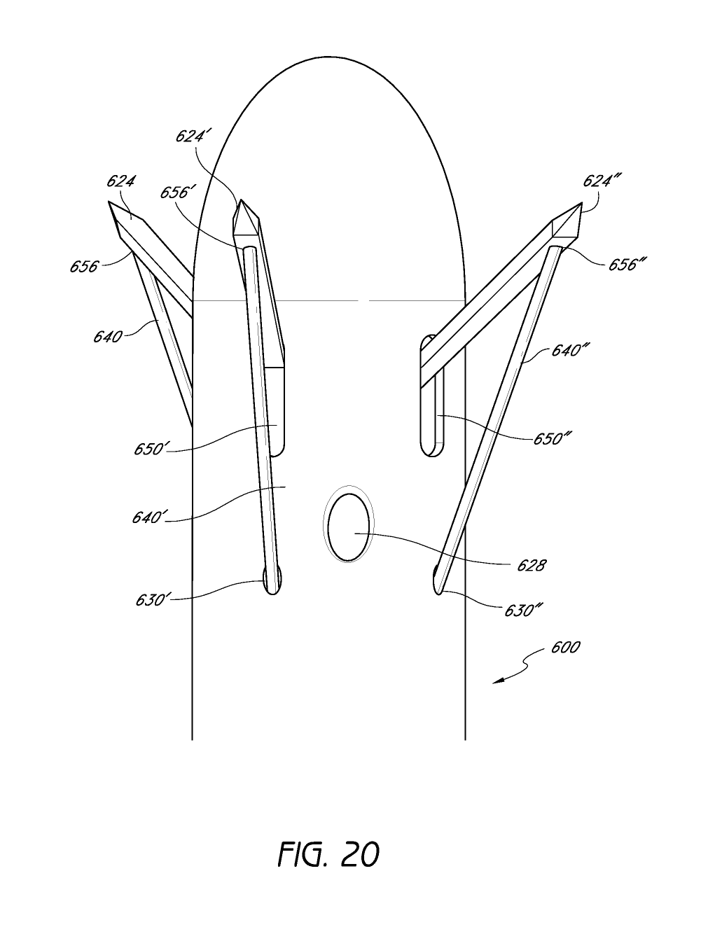

FIG. 19 illustrates an alternative embodiment of an arm 624 that may be used with any of the devices described above. The arm 624 can include a sharp end 682, an annular recess 680, a clasp 656, a slit 684, a hinge 690, a pin slot 692, and a hinge receiving portion 694. The hinge receiving portion 694 receives a hinge portion of the other arm 624′. The distal end of an actuating rod 658 (not shown) can be attached to the hinge portions of the arms 624-624′″ (FIG. 20) via a pin (not shown). Actuation of the actuating rod 658 controls the movement of the arms 624-624′″. As the arms 624-624′″ pivot outwardly, the sharp ends 682 of the arms advantageously pierce through tissue, such as through an outer or interior surface of the heart walls, which place the clasps 656 and annular recess 680 and the suture loop 641 within the heart tissue.

FIG. 20 illustrates a suturing device 600 similar to the device illustrated in FIGS. 15-16, but having the sharp arms 624 illustrated in FIG. 19. The sharp arms 624 provide a plurality of arms 624-624′″ the capability to deploy within the thick outer wall of the heart, rather than deploying internal to the heart outer wall. In some embodiments, the device 600 can include two, four, six, eight arms or more, or alternatively, an odd number of arms. Deploying the sharp arms 624 advantageously provides for the placement of sutures 52 within the heart outer wall, extending outwardly therefrom to the outside of the body, which allows for the sutures 652 and the needles 640 to avoid exposure or access to the internal cavity of the heart outer wall in their deployed condition.

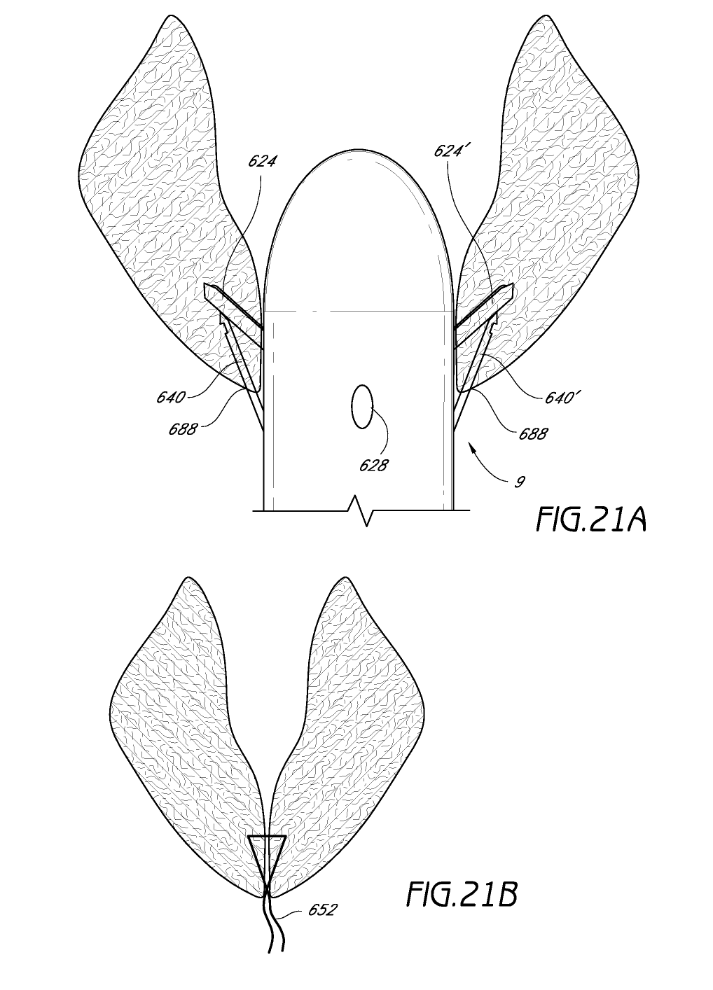

A method of using the suturing device 600 of FIG. 20 on a body structure, such as the heart outer wall, is illustrated in FIGS. 21A-21B. The physician initially advances the distal portion 600 of an elongate body 632 of the suturing device through the patient's body toward the desired body structure, such as the heart outer wall. In some embodiments, the elongate body 632 can have a constant diameter. The body structure can be accessed by various methods including: percutaneously, laparoscopically, or through an incision in general open surgery. During the insertion of the suturing device, the elongated body 632 can be articulated and rotated relative to the main body in order to steer the distal portion through the body structure.

When used on a transapical opening, once the physician places the distal portion 600 of the suturing device at the location within transapical opening 9, the arm trigger 104 (see FIG. 2A) or trigger actuator 308 (see FIG. 12) can be depressed to advance the arms 624 out of the arm apertures 650, 650′ as shown in FIG. 21A. As the arms 624 are advanced outward and become fully deployed, the distal portion of each arm 624 penetrates the tissue of the heart outer wall. As the arms 624 penetrate the heart tissue, the end portions of the suture 652 can be inserted into the tissue as shown in FIG. 21A.

After the arms are fully deployed, the physician pulls, or squeezes, the trigger actuator 308 proximally relative to the handle 310 to advance the needles 640, 640′ through the needle apertures and out toward the clasps 656 of each arm as shown in FIG. 21A. As each needle is advanced, the needle can pierce the tissue of the body structure at a location proximal to the location where the arm pierced the tissue. The needles continue to advance through the tissue until they engage the loops 641, 641′ at the ends of the suture. The needles can be automatically retracted by the handle 302, or alternatively the handle 101, relative to the main body, thereby removing the suture from the needle receiving portion of each arm and drawing the suture 652 ends back toward the suturing device 600 as shown in FIG. 21B. It should be noted that each suture end portion can be inserted into the tissue by an arm along a first path and then retracted from the tissue by a needle along a second path.

After the suture has been applied through the tissue of the biological structure, the arms can be retracted depressing the release trigger. The arms can be retracted so that the suturing device can be removed from the biological structure of the heart without damaging the surrounding tissue. The physician removes the suturing device from the biological structure with the suture loops 641 still held by the needles. If necessary, this procedure can be repeated to insert multiple sutures through, or within, the outer walls of the heart. After the suture(s) are in place, the end portions of each suture can be drawn together to create tension and pull the walls of the heart into contact with each other as shown in FIG. 21B. The suture 652 ends can be secured together with a securement, such as a knot as described below in connection with FIGS. 24-26, to close the biological structure.

In one embodiment, the suturing device advantageously incorporates arms 624 that penetrate the walls of the heart at an acute angle relative to the elongate body 632. When the arms 624 are in their fully extended position, they form an angle relative to each other that can be less than 180°, about 90°. The “forward-firing” arms 624 of the suturing device 600 embodiment can be particularly advantageous for penetrating heart outer wall-shaped tissue structures. The angle of the arms 624 enables the needles 640 to penetrate deeply into the tissue, thereby allowing the suture 652 to grab more tissue and form a stronger connection. The angle also enables the arms 624 to penetrate difficult to reach locations.

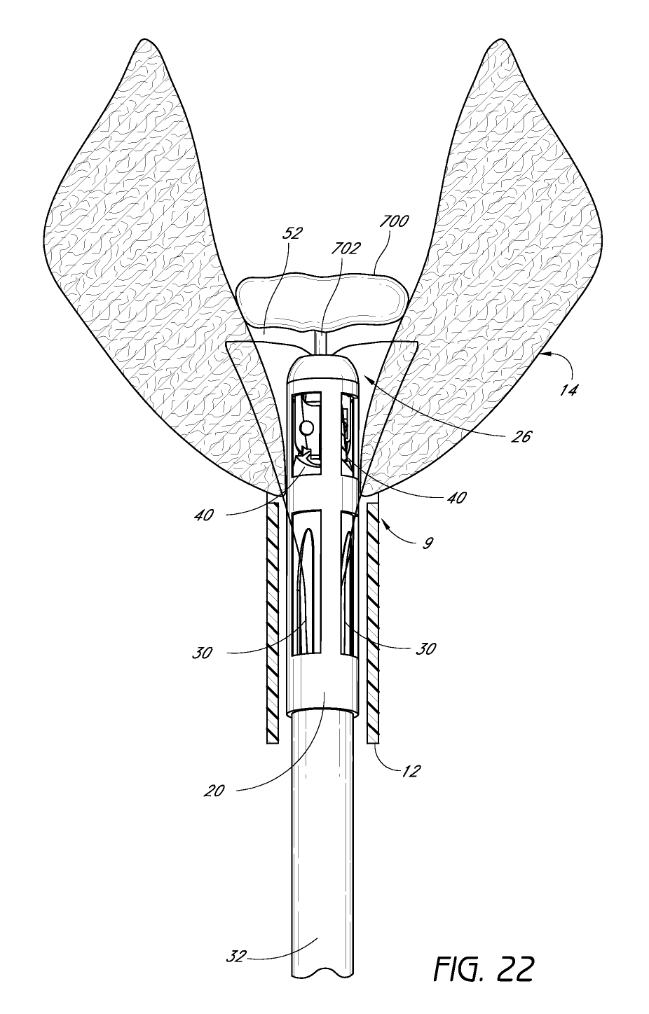

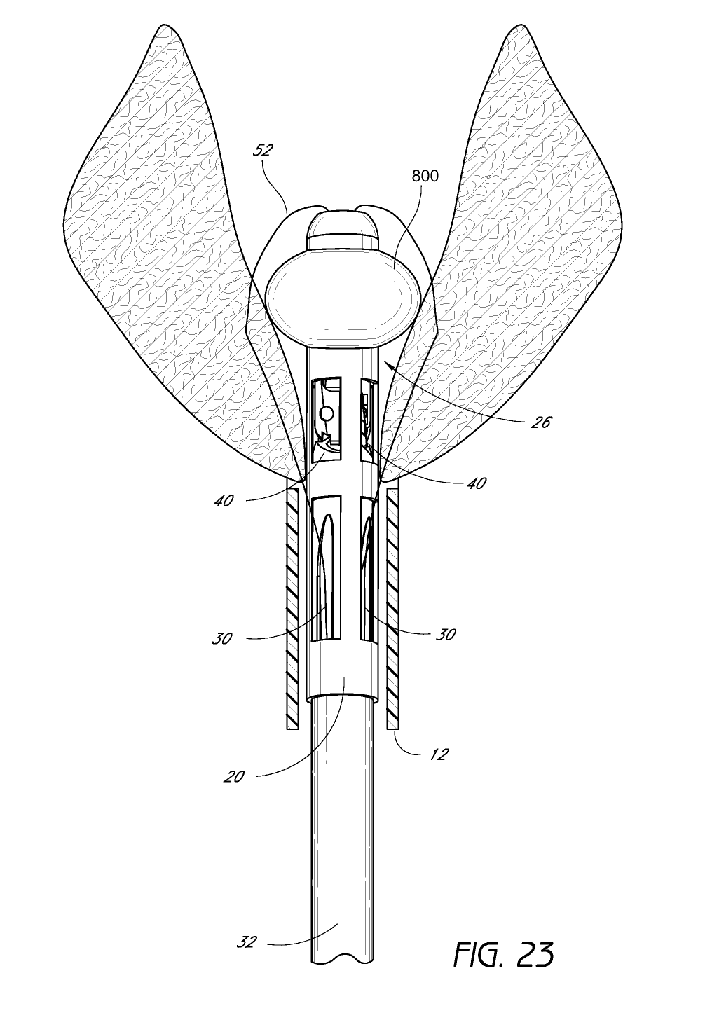

FIGS. 22-23 illustrate occlusion devices 700, 800 that can be used with the suturing devices described above to temporarily occlude the opening 9 and minimize the amount of blood escaping from the heart through the opening 9. In another embodiment, the occlusion devices 700, 800 can be adapted to occlude the entire heart cavity, including the opening 9.

For purposes of illustration, the occlusion devices 700, 800 are shown in FIGS. 22-23 in use with the suturing device of FIGS. 3A-3C. Alternatively, the occlusion devices 700, 800 can be adapted for use with other suturing devices including, for example, any of the suture devices described above with reference to FIGS. 1-21.

In FIG. 22, the occlusion device can include a balloon 700 which can be adapted to temporarily occlude the opening 9 to be sutured. The balloon 700 can comprise polyethylene, polyurethane, other polymers or any other material with similar properties. The balloon 700 can be attached to a hollow tube 702 which can be attached to a lumen (not shown) within the distal end 20 and the elongate body 32. Alternatively, the hollow tube 702 can extend through the lumen within the distal end 20 and the elongate body 32, and can slide within such lumen. The hollow tube 702 can be flexible or substantially rigid. The hollow tube 702 can be used to inflate the balloon 700. The balloon 700 can be inflated with saline solution or any fluid that is safe for internal occlusion devices.

In operation, inflation of the balloon 700 can be initiated after (1) the needles 40 capture the ends of the suture 42 from the arms 24, 24′ and (2) the arms 24, 24′ can be retracted into the distal end 20, as illustrated in FIG. 22. The balloon 700 temporarily occludes the opening 9 while the distal end 20 can be being withdrawn proximally from the tissue 14 and the physician can be tying a knot with the suture ends. The physician slides the knot distally toward the opening 9. Before the physician tightens the knot, the physician deflates the balloon 700 and withdraws the balloon 700 from the heart transapical opening 9. Finally, the physician tightens the knot to close the opening 9.

FIG. 23 illustrates an embodiment of the balloon 800, having similar characteristics to that of the balloon 700. The balloon 800 can be coupled to the external surface of the distal end 20 of the suturing device 100. The balloon itself typically defines an interior volume about an opening in an outer wall of the elongate body in communication with a lumen in the tube of the elongate body. Similar to the embodiment illustrated in FIG. 22, a hollow tube can extend from the distal end 20, except from a portion of the longitudinal wall rather than the hole 46 at the distal tip 54 (FIGS. 3A-3C), in order to communicate with the balloon 800. Like balloon 700, balloon 800 can be inflated with saline solution or any fluid that is safe for internal occlusion devices. Subsequently, the physician deflates the balloon 800 and withdraws the balloon 800 from the heart transapical opening 9 prior to tightening the knot to close the opening 9.

FIG. 24 illustrates one embodiment of a knot placement device 900 that can be used to apply a knot to the suture 52. The knot placement device 900 can include a handle 902 and a shaft 904 extending distally from the handle. The handle 902 can include an elongate tubular body extending from a proximal end to a distal end, and can include an actuator 906 and a distal end portion 910. The handle 902 can further comprise a cam 908 and a spring 912, shown in its rest position, disposed between the cam 908 and end portion 910. The actuator 906 can be a thumb or finger button in contact with the cam 908. End portion 910 can be fixedly attached to an outer tube 914 by glue, press fit, injection molding, or other suitable means known to one of ordinary skill in the art. An intermediate tube 916 can be concentrically and slidably disposed within the outer tube 914. A push rod 918 can be concentrically and slidably disposed within the intermediate tube 916 and fixedly attached to the cam 908. It should be appreciated that it is contemplated that the knot placement device 900 does not necessarily comprise an intermediate tube 916; however its inclusion provides certain benefits.

Depression of the actuator 906 causes the cam 908 to move distally, compressing the spring 912, thereby moving the push rod 108. After traveling for a certain desired distance, the cam 908 engages a proximal end of the intermediate tube 916, causing the intermediate tube 916 to also move distally. Upon release of the actuator 906, the spring 912 expands to move the cam 908 and the push rod 918 proximally. In the illustrated embodiment, the intermediate tube 916 can be freely slidable over the push rod 918.

In one embodiment, not shown, the cam 908 can include a detent in the surface which contacts the actuator 906. The detent can signal to the user a specific degree of advancement of the push rod 918, the intermediate tube 916, or both. For example, the detent can signal that the push rod has been advanced sufficiently far to insert the plug into the knot body, as described below. The detent can also indicate travel up until, but not including, the point at which the cam 908 engages the intermediate tube 916. The detent can be shaped so as to prevent the actuator 906 from returning to its original position. The cam can comprise multiple detents to indicate multiple increments of travel. To return the actuator to its initial position, the actuator and cam can include a mechanism such that after the actuator can be fully depressed, the actuator can automatically return to its initial position. Alternatively, the actuator can have a locked configuration, either at one of the detents or in a fully depressed configuration, and the handle can include a mechanism by which a second actuator can be used to release the cam and actuator to return to their initial positions.

In one embodiment, not shown, the intermediate tube 916 can comprise a keyway and the outer tube 914, the end portion 910, or both can comprise a key. Alternatively, the intermediate tube 916 can comprise a key and the outer tube 914, the end portion 910, or both can comprise a keyway. Providing such a key and keyway can be used to keep the intermediate tube 916 aligned with the outer tube. Other embodiments are contemplated to maintain rotational alignment of the intermediate tube, such as rotationally fixing the intermediate tube relative to the push rod. Providing such a key and keyway can also be used to constrain the range of sliding movement of the intermediate tube 916.

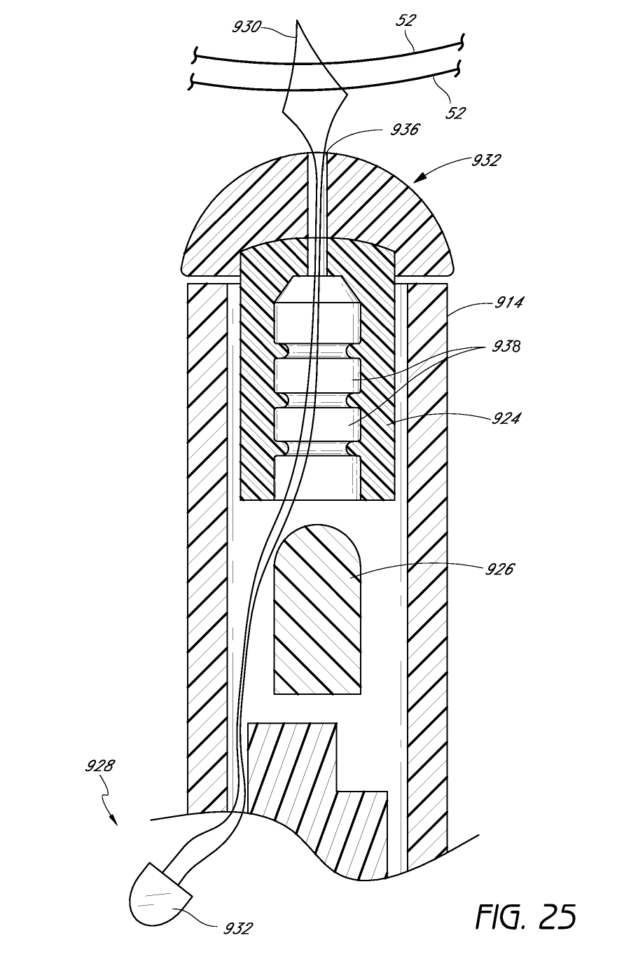

As shown in FIG. 25, a knot, comprising a knot body 924 and a plug 926, can be disposed within the outer tube 914 at its distal end. In another embodiment, the knot body can include an atraumatic tip 932. The tip 932 can be rounded and have an outer diameter about the same as that of the outer tube 914. The tip can also include a flat transition 934 as well. The tip 932 can be integrally formed with the knot body 924 or can be separately attached. As illustrated, the tip 932 can have an aperture 936 extending axially through the tip, opening to the cavity inside the knot body. When the knot is delivered into a patient as described above, the atraumatic tip prevents damage to the patient.

Alternatively, the fit between the knot body 924 and the outer tube 914 can not retain the knot body 924 in the outer tube 914. The knot body 924 can be at the distal end of the outer tube 914, and can protrude slightly distal to the distal end of outer tube 914. The plug 926 can be positioned proximal to the knot body 924, and can be slidably disposed within the intermediate tube 916, having a distal end located proximally from the knot body and distally from the push rod 918. The plug 926 has an outer dimension configured to be inserted into an inner cavity of the knot body 924. The intermediate tube 916 can be sized and positioned such that its distal end can abut knot body 924.

As shown in FIG. 25, the outer tube 914 can include a side hole 920 near its distal end. The intermediate tube 916 can include a slot (not shown) extending proximally from its distal end, forming a C-shaped cross section. At a proximal end of the slot, a sharpened cutting surface can be provided to cut suture 52, as described below. The slot can also be spaced from the distal end of the intermediate tube, such that the distal end of the tube still forms a complete circle in cross-section. The outer tube 914, intermediate tube 916 and push rod 918 can be made of any suitable material, including but not limited to metals, plastics, and a combination of metals and plastics.

As shown in FIG. 25, in a preloaded configuration, the knot placement device 900 can include a threader 928 comprising a tab 931 and a looped wire 930 passing through the side hole 920 in the outer tube 914. The wire 930 extends through the slot 122 located in the intermediate tube 916, and through knot body 924, exiting through opening 936 at the distal end of the knot body 924. The threader 928 can be used to load the suture into the knot placement device as described below. The threader 928 also prevents the knot body 924 from escaping from the placement device 900 when the knot body can be provided with an outer dimension of the same or smaller size than the inner wall of the outer tube 914.

With reference to FIG. 25, the knot body 924 can be generally tubular and comprise a proximal end, a distal end, and a longitudinal axis. The knot body 924 further defines an inner cavity and can include an opening 936 at its distal end. The knot body can be of a generally constant inner diameter and outer diameter. Alternatively, the inner diameter, the outer diameter, or both can generally taper along the longitudinal axis of the knot body. Alternatively, the inner diameter, the outer diameter, or both can generally taper along a portion of the longitudinal axis and can be of a generally constant inner diameter, outer diameter or both over a portion of the longitudinal axis.

The opening 936 at the distal end of the knot body can, in some embodiments, be of a reduced diameter relative to an inner cavity of the knot body 924. The knot body also can include an opening at the proximal end. The opening at the proximal end can, in some embodiments, be of a reduced diameter relative to an inner cavity of the knot body 924. The knot body can further comprise protrusions 938 extending from the inner surface of the knot body 924 toward the longitudinal axis. Protrusions 938 can be formed as rings as illustrated, or as spirals, spikes, bumps, or other suitable structures or combinations of structures.

Referring to FIG. 25, in one embodiment, the knot body 924 can be located distally from the plug 926 within the outer tube 914. The plug can be sized to be inserted into the inner cavity of the knot body 924, and can have a tapered configuration. Alternatively, the plug 926 can have a constant cross-section over a majority of its length, with a tapered, chamfered or rounded distal end for facilitating insertion into the knot body 924. The outer dimension of the plug 926 can be slightly larger than the inner dimension of the cavity of the knot body 924, such that when the plug is inserted into the cavity, a relatively secure fit can be provided between the two. The protrusions 938 within the knot body further facilitate the relative securement. The plug 926 can also comprise indentations, not shown, for receiving the protrusions 938 to secure the plug 926 more surely in the knot body 924. Other embodiments are contemplated wherein protrusions can be formed on the plug 926 with or without indentations formed in the inner cavity of the knot body 924. It is also contemplated that in some embodiments both the plug 926 and the knot body 924 can comprise protrusions and indentations, respectively. In certain embodiments, insertion of the plug 926 into the knot body 924 can cause the knot body 924 to slightly expand. Both the knot and the knot body can be formed of any suitable resilient materials, and in one embodiment, can be made from the same material as the suture, more preferably polypropylene.

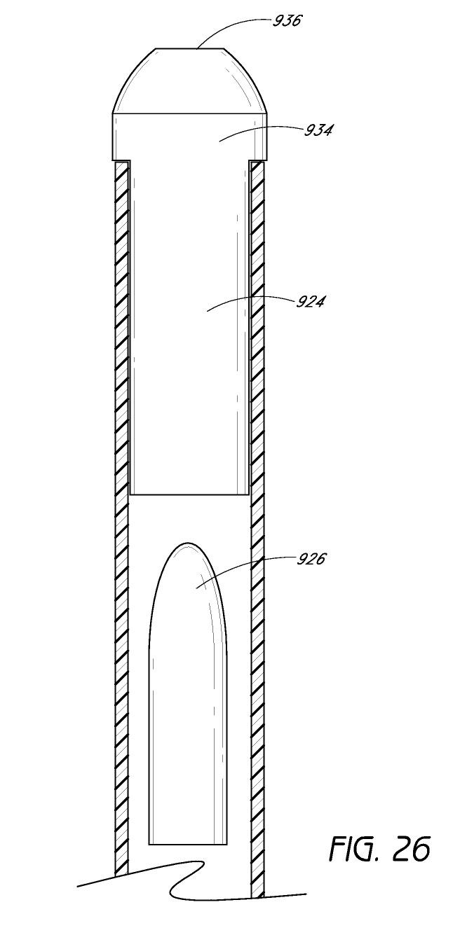

FIGS. 25-26 illustrate one embodiment for placing a knot utilizing the knot placement device 900 described above. A pair of sutures ends 52 can be passed through the loop 930 of threader 928. The threader can be preloaded into the knot placement device 900 as described above. The tab 931 of threader 928 can be pulled proximally to dispose suture 52 in the device. Suture 52 can be held in tension, by hand or otherwise, while the device 900 can be advanced until the knot body 924 or shaft 904 contacts at least one tissue portion. The actuator 906 can be depressed to advance the push rod 918, thereby forcing the plug 926 distally into the knot body 924 and trapping suture 52 there between the plug 926 and the knot body 924. The actuator can be further depressed until the cam 908 contacts the proximal end of intermediate tube 916, causing the intermediate tube 916 to contact knot body 924 and eject the knot from the shaft 904. Advancement of intermediate tube 916 can also cause cutting surface to sever suture 52 where it extends out of opening. The knot placement device can then be removed, leaving the knot in place against the tissue portions.

In one embodiment, the knot can be ejected from the shaft 904 while leaving the sutures 52 un-severed. For example, the knot can be ejected before the cutting surface reaches the suture 52. In another embodiment, no intermediate tube can be provided, and the suture can be cut manually.

In an embodiment including the intermediate tube, the device 900 can be configured such that the distal ends of the outer tube 914, intermediate tube 916, and the push rod 918 lie generally flush relative to one another and can be held relatively in position. This position can be held, for example, by depressing the actuator until it rests in a detent in cam 908. The detent can signal to the user that the plug 926 has been inserted into knot body 924, but also that the sutures 52 have not been cut. At such time, the placement device can be used to further advance the knot against tissue portions using the distal end surface of the shaft. The actuator can be further depressed to advance the push rod 918 and intermediate tube 916 to sever sutures 52.

The actuator 906 and cam 908 can also be provided with locking mechanisms that prevent the actuator 906 from returning to its original position. Further details are provided in U.S. Patent Application Publication No. 2006/0069397, published on Mar. 30, 2006, the entirety of which is hereby incorporated by reference herein. Such an embodiment can be advantageous to hold the push rod flush with the distal end of the outer tube to provide a surface that can be utilized to further advance and position the knot against tissue portions.

It will be appreciated that other embodiments can be contemplated without use of the intermediate tube, but can still be capable of severing the suture. For example, the push rod can be provided with portions of differing diameter. A distal, smaller diameter can be sized to engage the plug 926 to push the plug into the knot body 924. A proximal, larger diameter can be provided on the push rod, which can include a sharpened surface at the transition between the larger and smaller diameter sections. Once the smaller portion of the push rod pushes the plug 926 into the knot body 924, the larger portion of the push rod can engage the knot body 924 to push the knot out of the placement device, while the sharpened surface on the push rod can sever the suture.

In the embodiment described above, when the knot body 924 and the plug 926 as described above are secured together, suture portions extending through the inner cavity of the knot body will be fixedly secured therein, forming a knot. It will be appreciated that many other embodiments can be possible for forming a knot, including various other shapes and configurations for the knot body and plug, as well as embodiments wherein only one component can be used to provide securement relative to a suture. It will also be appreciated that in those embodiments in which the knot can include a knot body and plug, the plug can be located within the shaft proximally from the knot body or the knot body can be located within the shaft proximally from the plug.

In any of the above-described methods, suture(s) can be placed through the tissue near the opening before or after performing another procedure or procedures through the opening. In some embodiments suture(s) can be placed both before and after performing one or more other procedures.

It is envisioned that the suturing devices and methods described herein can be used to close or reduce a variety of tissue openings, lumens, hollow organs or natural or surgically created passageways in the body. These include, but are not limited to, arterial openings or other blood vessel openings, septal defects, patent foramen ovale, and heart valves. The devices and methods can also apply multiple sutures or other pieces of material across the opening simultaneously.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. For example, the embodiments disclosed above can be used to place suture in a variety of biological tissue locations, and may include numerous combinations of extending arms, needles and actuation mechanisms. In addition, while a number of variations of the invention have been shown and described in detail, other modifications, which are within the scope of this invention, will be readily apparent to those of skill in the art based upon this disclosure. It is also contemplated that various combinations or subcombinations of the specific features and aspects of the embodiments can be made and still fall within the scope of the invention. Accordingly, it should be understood that various features and aspects of the disclosed embodiments can be combined with or substituted for one another in order to form varying modes of the disclosed invention. Thus, it is intended that the scope of the present invention herein disclosed should not be limited by the particular disclosed embodiments described above, but should be determined only by a fair reading of the claims that follow.

What is claimed is:

1. A suturing apparatus configured to suture an opening made through an outer wall of the heart, comprising:
an elongate body having a proximal end and a distal end, a handle at the proximal end of the elongate body configured to be manipulated from outside of the heart, wherein the elongate body has a substantially constant outer diameter between the handle and the distal end;
a tapered or rounded tip positioned distal to the distal end of the elongate body and configured to be delivered through the opening and into an interior of the heart;
a plurality of arms proximal to the tip arranged symmetrically about the outer diameter of the elongate body, the plurality of arms configured to hold portions of suture a distance away from the outer diameter of the elongate body, the plurality of arms being extendable from said body from a retracted position to an extended position, wherein the plurality of arms in the extended position point distally and form an acute angle with a longitudinal axis of the elongate body;
a plurality of needles slidably housed in said elongate body, the plurality of needles being movable along the longitudinal axis of the elongate body and outwardly from the body to pass through heart tissue into engagement with the suture portions held by the plurality of arms, the plurality of needles further being retractable away from the plurality of arms back through the heart tissue to draw the suture portions through the heart tissue.

2. The suturing apparatus of claim 1, wherein the arms have a sharp tip configured to pierce heart tissue.

3. The suturing apparatus of claim 1, wherein the needles are moveable in a proximal-to-distal direction to engage the suture portions held by the arms.

4. The suturing apparatus of claim 1, wherein the needles are moveable in a distal-to-proximal direction to engage the suture portions held by the arms.

5. The suturing apparatus of claim 1, wherein the plurality of arms each forms an angle of between about 40 and 60 degrees with the longitudinal axis of the elongate body.

6. The suturing apparatus of claim 1, wherein the plurality of arms extend a distance of about 1 mm to 15 mm away from the outer diameter of the elongate body.

7. The suturing apparatus of claim 1, comprising two arms and two needles.

8. The suturing apparatus of claim 1, comprising four arms and four needles.

9. The suturing apparatus of claim 1, wherein the handle comprises an actuator configured to extend the arms and a trigger configured to move and retract the needles.

10. A method of performing a procedure within the heart; comprising:
forming an opening through the outer wall of the heart to provide access to an interior of the heart;
delivering a treatment instrument through the opening;
performing a procedure on the heart with the treatment instrument; and
closing the opening using a suturing apparatus comprising:
an elongate body having a proximal end and a distal end, a handle at the proximal end of the elongate body configured to be manipulated from outside of the heart, wherein the elongate body has a substantially constant outer diameter between the handle and the distal end;
a plurality of arms proximal to the tip arranged symmetrically about the outer diameter of the elongate body, the plurality of arms configured to hold portions of suture a distance away from the outer diameter of the elongate body, the plurality of arms being extendable from said body from a retracted position to an extended position, wherein the plurality of arms in the extended position point distally and form an acute angle with a longitudinal axis of the elongate body;
a plurality of needles slidably housed in said elongate body, the plurality of needles being movable along the longitudinal axis of the elongate body and outwardly from the body to pass through heart tissue into engagement with the suture portions held by the plurality of arms, the plurality of needles further being retractable away from the plurality of arms back through the heart tissue to draw the suture portions through the heart tissue.

11. The method of claim 10, wherein the opening is formed in an apex of the heart.

12. The method of claim 10, wherein the treatment instrument comprises a valve repair device, and performing the procedure comprises repairing the valve.

13. A method of closing a transapical opening in a wall of the heart, comprising:
delivering a suturing device through the transapical opening, the suturing device comprising an elongate body having a proximal end and a distal end;
extending at least one arm from the suturing device from a retracted position to an extended position, the at least one arm holding a portion of suture;
advancing at least one needle through heart tissue adjacent the transapical opening into engagement with the suture portion held by the arm;
retracting the at least one needle through the heart tissue adjacent the transapical opening to draw the suture portion through the heart tissue; and
closing the transapical opening with the suture portion.

14. The method of claim 13, further comprising:
extending a plurality of arms from the suturing device from a retracted position to an extended position, the plurality of arms holding portions of suture, wherein the plurality of arms hold portions of suture;
advancing a plurality of needles through the heart tissue adjacent the transapical opening into engagement with the suture portions held by the plurality of arms;
retracting the plurality of needles through the heart tissue adjacent the transapical opening to draw the suture portions through the heart tissue; and
closing the transapical opening with the suture portions.

15. The method of claim 13, wherein the at least one arm is extended alongside an outer wall of the heart, and the at least one needle is advanced from inside the heart in a distal-to-proximal direction.

16. The method of claim 15, wherein the at least one arm when extended points distally and forms an acute angle relative to a longitudinal axis of the elongate body.

17. The method of claim 13, wherein the at least one arm is extended alongside an inner wall of the heart, and the at least one needle is advanced from outside the heart in a proximal-to-distal direction.

18. The method of claim 17, wherein the at least one arm when extended points distally and forms an acute angle relative to a longitudinal axis of the elongate body.

19. The method of claim 13, wherein the at least one arm is extended into heart tissue adjacent the transapical opening.

20. The method of claim 13, wherein the at least one needle extends outwardly away from the elongate body when being advanced through heart tissue.

21. The method of claim 13, wherein the suturing device is delivered over a guidewire through the transapical opening.

22. The method of claim 13, further comprising positioning a balloon against the transapical opening after drawing the suture portion through the heart tissue.
